(19) United States
(12) Patent Application Publication
(10) Pub. No.: US 2005/0009052 A1
(43) Pub. Date: Jan. 13, 2005

(54) Differential tag length analysis of cell proliferation

(75) Inventors: Frantisek Supek, San Diego, CA (US); Nicholas Gekakis, San Diego, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: IRM LLC, a Delaware Limited Liability Company, Hamilton (BM)

(21) Appl. No.: 10/830,708

(22) Filed: Apr. 22, 2004

Related U.S. Application Data
(60) Provisional application No. 60/465,019, filed on Apr. 22, 2003.

(51) Int. Cl. C12Q 1/68; C12P 19/34

(52) U.S. Cl. 435/6; 435/91.2

(57) ABSTRACT

The present invention is based on a novel cell tagging approach called “Differential Tag Length” method (DTLA) that allows the quantitative analysis of a mixture of multiple cell types (e.g., strains) grown in very small cultivation volumes. DTLA can also be fully automated and adapted for a high-throughput format. The invention provides methods for detecting proliferation of a mixture of cell types in a culture, for screening a library of compounds to identify those compounds that modulate proliferation of a cell, and for detecting the presence or absence of targets in a sample. The invention also provides kits comprising at least first polynucleotide tag and a second polynucleotide tag.

### CROSS-REFERENCES TO RELATED APPLICATIONS

This application claims the benefit of U.S. Provisional Patent Application No. 60/465,019, filed Apr. 22, 2003, the disclosure of which is incorporated by reference in its entirety for all purposes.

### STATEMENT AS TO RIGHTS TO INVENTIONS MADE UNDER FEDERALLY SPONSORED RESEARCH AND DEVELOPMENT

Not applicable.

### BACKGROUND OF THE INVENTION

The measurement of cell growth is often used as the starting point in the discovery of novel gene function as well as the identification of biologically active organic molecules. Under specific conditions (e.g. increased temperature or presence of an inhibitor), a mutant lacking the studied gene can exhibit resistance to the treatment or fail to grow (see, e.g., Smith, et al. (1996) Science 274, 2069-2074; Huang and Schreiber (1997) Proc. Natl. Acad. Sci. USA 94, 13396-401; Rieger et al. (1999) in Methods in Microbiology 28, eds. Craig, A. G. & Hoheisel, J. D. (Academic Press, London), pp. 205-228; Young et al. (1998) Nat. Biotechnol. 16, 946-950; and Sasseti et al. (2001) Proc. Natl. Acad. Sci. USA 98, 12712-12717). Frequently, such phenotypes may be indicative of the biochemical function of the knocked-out gene product and/or the identity of the protein target. This approach is widely applied to the budding yeast Saccharomyces cerevisiae and its use has been further stimulated by the comprehensive set of S. cerevisiae gene deletion mutants (see, e.g., Giaever, G. et al. (2002) Nature 418, 387-391; Steinmetz, L. M. et al. (2002) Nat. Genet. 31, 400-404; Birrell et al. (2001) Proc. Natl. Acad. Sci. USA 98, 12608-12613; Kumar et al. (2002) Nat. Biotechnol. 20, 58-63; and Ni and Snyder (2001) Mol. Biol. Cell 12, 2147-2170). Concurrent with the ongoing characterization of S. cerevisiae deletion strains, an even larger number of strains will need to be constructed in order to examine the synthetic phenotypes (e.g. synthetic lethality, epistasis) of all double gene knockouts (see, e.g., Tong et al. (2001) Science 294, 2364-2368). Instead, a highly specific small molecule ligand can also be used to alter the function of one gene product from the pair directly. Small molecule inhibitors, although laborious to identify, have some advantages when compared with gene knockouts. They allow the modification of biochemical activity of a protein in an adjustable manner and are frequently active against its target in multiple species (see, e.g., Shogren-Knaak et al. (2001) Annu. Rev. Cell Dev. Biol. 17, 405-433). Such protein-binding molecules have been instrumental in elucidating the molecular details of actin dynamics (latrunculin A) (see, e.g., Spector et al. (1983) Science 219, 493-495; Coué et al. (1987) FEBS Lett. 213, 316-318; Ayscough et al. (1997) J. Cell Biol. 137, 399-416; and Morton et al. (2000) Nat. Cell Biol. 2, 376-378), N-glycosylation (tunicamycin) (see, e.g., Kuo and Lampen (1974) Biochem. Biophys. Res. Commun. 58, 287-295; Barnes et al. (1984) Mol. Cell. Biol. 4, 2381-2318; and Su et al. (1999) FEBS Lett. 453, 391-394), nutrient starvation signaling (rapamycin) (see, e.g., Barbet et al. (1996) Mol. Biol. Cell. 7, 25-42; Beck and Hall (1999) Nature 402, 689-692; and Hardwick et al. (1999) Proc. Natl. Acad. Sci. USA 96, 14866-14870), and other cellular processes (see, e.g., Hung et al. (1996) Chem. Biol. 8, 623-639). Recent advances in combinatorial chemistry have dramatically increased the likelihood of finding small molecules with desirable properties. Their identification is further facilitated by the development of cell-based screens for inhibitor targets in a few microorganisms (S. cerevisiae, Candida albicans, and E. coli) (see, e.g., Giaver et al. (1999) Nat. Genet. 21, 278-283; De Backer, M. D. et al. (2001) Nat. Biotechnol. 19, 235-241; and DeVito, J. A. et al. (2002) Nat. Biotechnol. 20, 478-483). These screens are based on the observation that lowering the expression of target protein results in an increased sensitivity to an inhibitor relative to the wild-type strain.

The limiting step in all of the above-described strategies is the number of growth assays that can be physically performed. This problem is especially acute when screening the libraries of organic molecules produced by combinatorial chemistry when only small quantities of compounds are available. One alternative is to grow all interrogated strains in a mixture and perform a post-growth strain composition analysis. This pooling approach has been applied for inhibitor targets determination with a set of S. cerevisiae deletion strains (see, e.g., Chen et al. (1995) Biotechniques 19, 744-746). The method, which relies on unique barcode sequences incorporated into the genome of deletion strains, can be scaled up to a few thousand strains in the mixture, but is not adaptable to high-throughput screens and the cost per assay is relatively high.

Thus, there is a need in the art for methods for quantitative analysis of mixture s of multiple types of cell. There is also a need for such methods that are also adaptable to high-throughput screening. The present invention fulfills this and other needs.

### BRIEF SUMMARY OF THE INVENTION

One embodiment of the present invention provides a method for determining the relative prevalence of each of two or more cell types in a mixture of cell types. For example, the methods are useful for detecting proliferation of a mixture of cell types in a culture. At least one cell of a first cell type and at least one cell of at least a second cell type is provided. The first cell type comprises a first polynucleotide tag and the second cell type comprises a second polynucleotide tag. Each polynucleotide tag comprises a first primer binding site on a first strand and a second primer binding site on a second strand, wherein the second strand is complementary to the first strand. Each polynucleotide tag also comprises a unique restriction site at a preselected position relative to the first primer binding site, wherein the position of the unique restriction site in the first polynucleotide tag differs from the position of the unique restriction site in the second polynucleotide tag. A primer that hybridizes to the first primer binding site on the first polynucleotide tag also hybridizes to the first primer binding site on the second polynucleotide tag and a primer that hybridizes to the second primer binding site on the first polynucleotide tag also hybridizes to the second primer binding site on the second polynucleotide tag.

The cells are incubated in a medium and under conditions suitable for proliferation of the cells. Proliferation of the cells is detected by (1) amplifying the polynucleotide tags with a pair of primers that hybridize to the first and second primer binding sites, thereby producing amplified product corresponding to any polynucleotide tags that are present; (2) cleaving the amplified products with a restriction enzyme that cleaves the amplified products at the unique restriction site, thereby forming cleaved amplification products; and (3) detecting the cleaved amplification products. The amount of a first cleaved amplification product corresponding to the first polynucleotide tag is correlated with the proliferation of the first cell type and the amount of a second cleaved amplification product corresponding to the second polynucleotide tag is correlated with the proliferation of the second cell type.

In some embodiments, the cells are yeast cells, plant cells, mammalian cells, and the like. For example, in some embodiments, the cells are cancer cells. The method can involve performing the amplification on a sample of purified genomic DNA obtained from the cells. Alternatively, the method can involve performing the amplification on a cell lysate.

In some embodiments, the cells express a pair of interacting proteins and cell proliferation is dependent upon the interactions between the interacting proteins.

In some embodiments, the polynucleotide tags are integrated into the genomes of the first and second cell types. At least one primer in the pair of primers is labeled, in some embodiments of the invention; in some embodiments, both primers are labeled. Suitable labels include, for example, a fluorescent label. In some embodiments, the second primer in the pair of primers comprises a 5′ phosphate and the method further involves contacting the amplified reaction products with an exonuclease that degrades a polynucleotide strand that comprises the primer that comprises the 5′ phosphate prior to contacting the amplified reaction products with the restriction enzyme.

In some embodiments, the relative prevalence and/or proliferation of at least 3, 5, 10, 15, 20, 25, 50, 75, or 100 cell types is detected. Each cell type includes a polynucleotide tag in which the position of the unique restriction site in the polynucleotide tag present in one cell type differs from the position of the unique restriction site in the polynucleotide tag present in other cell types. In some embodiments, the cells are in a well of a microtiter plate. For example, the microtiter plate can be a 96, 384 or 1536 well microtiter plate.

In some embodiments, the cells are contacted with a potential modulator of cell proliferation. For example, the potential modulator of cell proliferation can be a small organic molecule. In some embodiments, at least one gene in at least a first cell type is expressed at a level that differs from expression of the gene in other cell types present. For example, cells of the first cell type can include a mutation that alters expression of the gene. As another example, cells of the first cell type comprise an expression construct from which is transcribed mRNA transcripts that correspond to those transcribed from the gene present in the genome of the cell, thereby causing the cells of the first cell type to comprise a higher level of mRNA transcripts that correspond to the gene than other cell types. Another example involves cells of the first cell type having a double-stranded RNA molecule that comprises a first polynucleotide sequence that is identical to a target region on the gene, and a second polynucleotide sequence that is complementary to the first polynucleotide sequence, wherein the double-stranded RNA molecule inhibits expression of the gene in cells of the first cell type.

Another embodiment of the present invention provides a method for screening a library of compounds to identify those compounds that modulate proliferation of a cell. At least one cell of a first cell type and at least one cell of at least a second cell type is provided. The first cell type comprises a first polynucleotide tag and the second cell type comprises a second polynucleotide tag. Each polynucleotide tag comprises a first primer binding site on a first strand and a second primer binding site on a second strand, wherein the second strand is complementary to the first strand. Each polynucleotide tag also comprises a unique restriction site at a preselected position relative to the first primer binding site, wherein the position of the unique restriction site in the first polynucleotide tag differs from the position of the unique restriction site in the second polynucleotide tag. A primer that hybridizes to the first primer binding site on the first polynucleotide tag also hybridizes to the first primer binding site on the second polynucleotide tag and a primer that hybridizes to the second primer binding site on the first polynucleotide tag also hybridizes to the second primer binding site on the second polynucleotide tag.

The cells are contacted with a compound, which can be a member of a library of compounds and incubated under conditions suitable for proliferation of the cells. Proliferation of the cells is detected by (1) amplifying the first and second polynucleotide tags in each vessel with a pair of primers that hybridize to the first and second primer binding sites, thereby producing amplified products corresponding to the polynucleotide tags that are present; (2) cleaving the amplified products with a restriction enzyme that cleaves the amplified products at the unique restriction site, thereby forming cleaved amplification products; and (3) detecting the cleaved amplification products, whereby the amount of a first cleaved amplification product corresponding to the first polynucleotide tag is correlated with the proliferation of the first cell type in the presence of the compound, and the amount of a second cleaved amplification product corresponding to the second polynucleotide tag is correlated with the proliferation of the second cell type in the presence of the compound. In some embodiments, the cells are present in a vessel and at least one test gene in at least a first cell type is expressed at a level that differs from expression of the test gene in other cell types present in the vessel. A change in the amount of the cleaved amplification product corresponding to the first cell type in one vessel compared to the amount of the cleaved amplification product corresponding to the first cell type in at least a second vessel indicates that the test gene in the first cell type encodes a gene product that is modulated by the compound that is present in the first vessel. For example, cells of the first cell type can have a mutation that alters expression of the test gene. Alternatively, cells of the first cell type can include an expression construct from which is transcribed mRNA transcripts that correspond to those transcribed from the test gene present in the genome of the cell, thereby causing the cells of the first cell type to comprise a higher level of mRNA transcripts that correspond to the test gene than other cell types. In other embodiments, cells of the first cell type comprise a double-stranded RNA molecule that comprises a first polynucleotide sequence that is identical to a target region on the test gene, and a second polynucleotide sequence that is complementary to the first polynucleotide sequence, wherein the double-stranded RNA molecule inhibits expression of the test gene in cells of the first cell type.

A further embodiment of the invention provides a mixture of cells comprising two or more cell types. Each cell type comprises a polynucleotide tag. Each polynucleotide tag comprises a first primer binding site on a first strand and a second primer binding site on a second strand. The second strand is complementary to the first strand. A primer that hybridizes to the first primer binding site on the first polynucleotide tag also hybridizes to the first primer binding site on the second polynucleotide tag; and a primer that hybridizes to the second primer binding site on the first polynucleotide tag also hybridizes to the second primer binding site on the second polynucleotide tag. Each polynucleotide tag also comprises a unique restriction site at a preselected position relative to the first primer binding site, wherein the position of the unique restriction site in the first polynucleotide tag differs from the position of the unique restriction site in the second polynucleotide tag. In some embodiments the mixture of cells comprises at least 5, 10, 15, 20, 25, 50, 75, or 100 cell types. In some embodiments, the cells are yeast cells, plant cells, mammalian cells, or other cell types. For example, in some embodiments the cells are cancer cells. In some embodiments, the cells express two interacting proteins (sometimes referred to as a bait protein and a prey protein), and proliferation of the cells is dependent upon the interaction between the proteins.

Yet another embodiment of the present invention provides a method for detecting the presence or absence of targets in a sample. A sample is contacted with at least a first and a second detection reagent. The first detection reagent comprises a binding moiety specific for a first target and a first polynucleotide tag. The second detection reagent comprises a binding moiety specific for a second target and a second polynucleotide tag. Each polynucleotide tag comprises a first primer binding site on a first strand and a second primer binding site on a second strand, wherein the second strand is complementary to the strand that comprises the first primer binding site. Each polynucleotide tag also comprises a unique restriction site at a preselected position relative to the first primer binding site, wherein the position of the unique restriction site in the first polynucleotide tag differs from the position of the unique restriction site in the second polynucleotide tag. A primer that hybridizes to the first primer binding site on the first polynucleotide tag also hybridizes to the first primer binding site on the second polynucleotide tag and a primer that hybridizes to the second primer binding site on the first polynucleotide tag also hybridizes to the second primer binding site on the second polynucleotide tag. The detection reagents that bind to a target in the sample are separated from detection reagents that do not bind to the target. The presence or absence of the bound first and second detection reagents is detected by: (1) amplifying the polynucleotide tags with a pair of primers that hybridize to the first and second primer binding sites, thereby producing amplified products corresponding to each polynucleotide tag present; (2) cleaving the amplified products with a restriction enzyme that cleaves the amplified products at the unique restriction site, thereby forming cleaved amplification products; and (3) detecting the cleaved amplification products, whereby the amount of the first cleaved amplification product is correlated with the amount of the first target and the amount of the second cleaved amplification product is correlated with the amount of the second target in the sample. In some embodiments, one or more of the targets comprise antigens and one or more of the binding moieties comprise antibodies. In other embodiments, the polynucleotide tags each comprise an open circle probe that is attached to the binding moiety by hybridization between the open circle probe and a target probe that is attached to the corresponding binding moiety.

Another embodiment of the invention provides a kit comprising at least a first polynucleotide tag and a second polynucleotide tag. Each polynucleotide tag comprises a first primer binding site on a first strand and a second primer binding site on a second strand. The second strand is complementary to the first strand. Each polynucleotide tag also comprises a unique restriction site at a preselected position relative to the first primer binding site, wherein the position of the unique restriction site in the first polynucleotide tag differs from that in the second polynucleotide tag. A primer that hybridizes to the first primer binding site on the first polynucleotide tag also hybridizes to the first primer binding site on the second polynucleotide tag and a primer that hybridizes to the second primer binding site on the first polynucleotide tag also hybridizes to the second primer binding site on the second polynucleotide tag. The kit also comprises a first primer that hybridizes to each of the polynucleotides at the first primer binding site and a second primer that hybridizes to a complementary strand of each of the polynucleotides at the second primer binding site. In some embodiments, the first primer comprises a detectable label. For example, the label can be a fluorescent label. In other embodiments, the second primer comprises a 5′ phosphate, in which case the kit can further include an exonuclease that can degrade polynucleotide strands that have a 5′ phosphate. In some embodiments, each polynucleotide tag is present in a vector that can integrate into a genome of a cell. For example, the vector can be an adenoviral vector, a retroviral vector, or a lentiviral vector. The kit can further include a restriction enzyme that cleaves the polynucleotide tags at the unique restriction site. In some embodiments, the kit comprises at least three polynucleotide tags in which the position of the unique restriction site in each polynucleotide tag differs from that in the other polynucleotide tags in the kit. In some embodiments, wherein the kit comprises at least ten polynucleotide tags in which the position of the unique restriction site in each polynucleotide tag differs from that in the other polynucleotide tags in the kit. In some embodiments, the first polynucleotide tag is attached to a first protein binding moiety and the second polynucleotide tag is attached to a second protein binding moiety. For example, the first protein binding moiety and the second protein binding moiety are antibodies in some kits of the invention. In some embodiments, the first polynucleotide tag comprises a first open circle probe and the second polynucleotide tag comprises a second open circle probe.

These and other embodiments of the invention are described in greater detail below.

### BRIEF DESCRIPTION OF THE DRAWINGS

FIG. 1 illustrates the differential tag length analysis principle. A unique restriction site (URS) is introduced into a DNA fragment at different positions to create the collection of tags. The tag DNAs are integrated into the genome of yeast strains and a mixture of such strains is grown in the absence or presence of an organic molecule inducing differential cell growth. At the end of cultivation, the tags are amplified by PCR with one fluorescent and one phosphorylated primer. The PCR product (indicated as a star) is cleaved at the unique restriction site and the fluorescent fragments are separated by capillary electrophoresis. The relative abundance of each strain within the mixture is reflected in the peak intensities of corresponding tags.

FIG. 2 illustrates data showing tag amplification rates during multiplex PCR. The starting stock of template DNA containing 11 tags (numbers 2, 4, 9, 12, 14, 16, 20, 26, 32, 38, and 42) was diluted 32× and 1,024×. FIG. 2A illustrates a capillary electrophoresis (CE) trace of reference PCR containing the highest template concentration. Peaks corresponding to tag signals are indicated with arrows. CE traces of 32-fold (FIG. 2B) and 1,024-fold (FIG. 2C) dilution of template DNA mixture are also shown. FIG. 2D shows changes in the percentage of peak areas expressed as function of number of amplification steps. Reference PCR is shown in blue. Black and white columns represent 32- and 1024-fold dilution of template DNA, respectively. The graph shows the average and standard deviation of four independent experiments.

FIG. 3 illustrates data demonstrating CE signal dependence on the amount of input tag DNA. An approximately equimolar mixture of 10 tag DNAs (tags 2, 4, 9, 12, 14, 16, 20, 26, 32, and 38) was supplemented with different amounts of tag 42 DNA and used as the template for PCR. Typical CE traces for mixtures containing 25% (FIG. 3A), 100% (FIG. 3B), and 250% (FIG. 3C) of tag 42 DNA (indicated with arrows) relative to other tags that are approximately equimolar are shown. (FIG. 3D) Tag 42 normalized signal intensities plotted vs. actual tag 42 DNA concentrations present in the PCR template. The graph shows the average and standard deviation of four independent experiments.

FIG. 4 illustrates data showing the comparison of growth of 11 yeast strains cultured individually and in mixture in the absence and presence of 1 μM staurosporine. Eleven yeast strains were grown separately with (FIG. 4A) or without (FIG. 4B) staurosporine. The following strains were used: wild-type, ARP2/arp2Δ, pdr5Δ/pdr5Δ, PKC1/pkc1Δ, pac10Δ/pac10Δ, ALG7/alg7Δ, gim4Δ/gim4Δ, rlm1Δ/rlm1Δ, pde2Δ/pde2Δ, bro1Δ/bro1Δ, and ACT1/act11Δ. Typical electropherograms of tags amplified from non-treated (FIG. 4C) and staurosporine-treated (FIG. 4D) mixed cultures. The arrows indicate the 4 staurosporine sensitive strains. (FIG. 4E) Relative strain composition of mixture without the addition of staurosporine at the beginning (black) and end of cultivation (white) without addition of staurosporine. (FIG. 4F). Comparison of strain composition at the end of cultivation without (black) and with (white) staurosporine. The arrows indicate strains that showed sensitivity to the inhibitor when grown individually.

FIG. 5 illustrates data showing changes in tag profiles of mixed yeast cultures grown in the presence of 8 mM caffeine (FIG. 5A), 2 μg/ml latrunculin B (FIG. 5B), and 0.4 μg/ml tunicamycin (FIG. 5C).

FIG. 6 summarizes tag profiles of mixed yeast cultures grown in the presence of caffeine, latrunculin B, staurosporine, and tunicamycin.

### DETAILED DESCRIPTION OF THE INVENTION

I. Introduction

The present invention is based on a novel cell tagging approach called “Differential Tag Length” method (DTLA) that allows the quantitative analysis of a mixture of multiple cell types (e.g., strains) by amplification and detection of unique oligonucleotide tags. Moreover, the invention allows for analysis of cells grown in very small cultivation volumes. In addition, DTLA allows for the detection and quantitative analysis of multiple targets in a sample. DTLA can also be fully automated and adapted for a high-throughput format.

One embodiment of the present invention provides a method for detecting proliferation of a mixture of cell types in a culture. A mixture of cell types is cultured under conditions in which the cells will proliferate. Each cell type has been transformed with a unique oligonucleotide. Thus, each cell type contains a unique oligonucleotide tag. Each unique tag contains a first primer binding site on a first strand and a second primer binding site on a second strand, wherein the first and second strands are complementary. Each unique tag also contains a unique restriction site at a preselected position relative to the first primer binding site. Even though the tags are unique, the same primer pair binds to all of the tags. To identify cells types that have proliferated, the oligonucleotide tags are amplified and the amplified products are detected. Only oligonucleotide tags from cell types that have proliferated will be amplified. Thus, detection of an amplified product corresponding to a unique oligonucleotide tag indicates that the cell type containing that particular oligonucleotide tag proliferated. Using methods known to those of skill in the art, (e.g., quantitative PCR) it is possible to quantitate the relative proliferation for each cell type in the mixture.

Another embodiment of the present invention provides a method for screening a library of compounds to identify those compounds that modulate proliferation of a cell. A mixture of cell types is contacted with a member of the library of compounds and cultured. Each cell type contains a unique oligonucleotide tag as described above. To identify cells types that have proliferated after contacting the library member, the oligonucleotide tags are amplified and the amplified products are detected. Only oligonucleotide tags from cell types that have proliferated will be amplified. Thus, detection of an amplified product corresponding to a unique oligonucleotide tag indicates that the cell type containing that particular oligonucleotide tag proliferated in the presence of the library member. Library members that enhance, inhibit, retard, or restore the ability of cells to proliferate are identified as modulators of cell proliferation.

Yet another embodiment of the present invention provides a method for detecting the presence or absence of targets in a sample. A binding moiety is linked to a unique oligonucleotide tag as described above. The binding moiety is contacted with a sample that contains or is suspected of containing at least one target to which the binding moiety specifically binds. To identify targets that are present in the sample, the oligonucleotide tags are amplified and the amplified products are detected. Only oligonucleotide tags linked to binding moieties that have specifically bound to their target will be amplified. Thus, detection of an amplified product corresponding to a unique oligonucleotide tag indicates that the specific target of a particular binding moiety is present in the sample. Using methods known to those of skill in the art, (e.g., quantitative PCR) it is possible to quantitate the relative amount of target in the sample.

II. Definitions

“Proliferation” as used herein refers to growth and/or division of cells. “Modulator of cell proliferation” as used herein refers to a compound that inhibits, enhances, stimulates, or retards cell proliferation.

“Polynucleotide tag” or “oligonucleotide tag” as used herein refers to an olignonucleotide sequence comprising a first primer binding site on a first strand and a second primer binding site on a second strand. The second strand is complementary to the first strand. The polynucleotide tags also comprise a unique restriction site at a preselected position relative to the first primer binding site. The position of the restriction site differs for each polynucleotide tag. One of skill in the art will appreciate that the particular restriction site, the particular nucleotide sequence of the oligonucleotide tag and the length of the oligonucleotide tags are not critical parts of the present invention. An oligonucleotide tag can be a sequence of any detectable length. The tags can comprise naturally occurring nucleotides, e.g., adenine (A), guanine (G), cytosine (C), and thymine (T), or synthetic nucleotides in any order. Typically, an oligonucleotide tag is at least 50, 75, 100, 125, 150, 175, or 200 nucleotides in length. In some embodiments, the polynucleotide tag comprises an open circle probe (see, e.g., U.S. Pat. No. 6,210,884).

“Binding moiety” as used herein refers to any moiety that specifically recognizes and/or binds to a target (i.e., a binding target). The binding may be either reversible or irreversible in a biological milieu. In some embodiments, the binding moiety is an antibody, a receptor, or a ligand.

An “amplification reaction” refers to any chemical reaction, including an enzymatic reaction, which results in increased copies of a template nucleic acid sequence. Amplification reactions include polymerase chain reaction (PCR) and ligase chain reaction (LCR) (see U.S. Pat. Nos. 4,683,195 and 4,683,202; PCR Protocols: A Guide to Methods and Applications (Innis et al., eds, 1990)), strand displacement amplification (SDA) (Walker, et al. Nucleic Acids Res. 20(7):1691 (1992); Walker PCR Methods Appl 3(1):1 (1993)), transcription-mediated amplification (Phyffer, et al., J. Clin. Microbiol. 34:834 (1996); Vuorinen, et al., J. Clin. Microbiol. 33:1856 (1995)), nucleic acid sequence-based amplification (NASBA) (Compton, Nature 350(6313):91 (1991), rolling circle amplification (RCA) (Lisby, Mol. Biotechnol. 12(1):75 (1999)); Hatch et al., Genet. Anal. 15(2):35 (1999); and U.S. Pat. No. 6,210,884) and branched DNA signal amplification (bDNA) (see, e.g., Iqbal et al., Mol. Cell Probes 13(4):315 (1999)).

“Amplifying” refers to submitting a solution to conditions sufficient to allow for amplification of a polynucleotide if all of the components of the reaction are intact. Components of an amplification reaction include, e.g., primers, a polynucleotide template, polymerase, nucleotides, and the like. Thus, an amplifying step can occur without producing a product if, for example, the template (e.g., the polynucleotide tag) is not present in the sample or is present in extremely low amounts. Likewise, an amplifying step can occur without producing a product if the primers are degraded.

“Amplification reagents” refer to reagents used in an amplification reaction. These reagents can include, e.g., oligonucleotide primers; borate, phosphate, carbonate, barbital, Tris, etc. based buffers (see, U.S. Pat. No. 5,508,178); salts such as potassium or sodium chloride; magnesium; deoxynucleotide triphosphates (dNTPs); a nucleic acid polymerase such as Taq DNA polymerase; as well as DMSO; and stabilizing agents such as gelatin, bovine serum albumin, and non-ionic detergents (e.g. Tween-20).

The term “primer” refers to a nucleic acid sequence that primes the synthesis of a polynucleotide in an amplification reaction. Typically a primer comprises fewer than about 100 nucleotides and preferably comprises fewer than about 30 nucleotides. Exemplary primers range from about 5 to about 25 nucleotides. The “integrity” of a primer refers to the ability of the primer to prime an amplification reaction. For example, the integrity of a primer is typically no longer intact after degradation of the primer sequences such as by endonuclease cleavage.

“Vessel” as used herein refers to any container that can be used to culture cells. Suitable containers include, for example, multi-well plates (e.g., 6, 12, 24, 28, 96, 384, or 1536 well microtiter plate), petri dishes, tissue culture tubes, flasks, roller bottles, and the like.

The terms “isolated,” “purified,” or “biologically pure” refer to material that is substantially or essentially free from components that normally accompany it as found in its native state. Purity and homogeneity are typically determined using analytical chemistry techniques such as polyacrylamide gel electrophoresis or high performance liquid chromatography. A protein that is the predominant species present in a preparation is substantially purified. The term “purified” denotes that a nucleic acid or protein gives rise to essentially one band in an electrophoretic gel. Particularly, it means that the nucleic acid or protein is at least 85% pure, more preferably at least 95% pure, and most preferably at least 99% pure.

The terms “nucleic acid” and “polynucleotide” are used interchangeably herein to refer to deoxyribonucleotides or ribonucleotides and polymers thereof in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

Unless otherwise indicated, a particular nucleic acid sequence also encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); Rossolini et al., Mol. Cell. Probes 8:91-98 (1994)). The term nucleic acid is used interchangeably with gene, cDNA, mRNA, oligonucleotide, and polynucleotide.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an α carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologues, and alleles of the invention.

The following eight groups each contain amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cysteine (C), Methionine (M) (see, e.g., Creighton, Proteins (1984)).

A “label” or “detectable label” is a composition detectable by spectroscopic, photochemical, biochemical, immunochemical, or chemical means. For example, useful labels include radioisotopes (e.g., ³H, ³⁵S, ³²P, ⁵¹Cr, or ¹²⁵I), fluorescent dyes, electron-dense reagents, enzymes (e.g., alkaline phosphatase, horseradish peroxidase, or others commonly used in an ELISA), biotin, digoxigenin, or haptens and proteins for which antisera or monoclonal antibodies are available (e.g., a polypeptide can be made detectable, e.g., by incorporating a radiolabel into the peptide, and used to detect antibodies specifically reactive with the peptide).

The term “subsequence” refers to a sequence of nucleotides that are contiguous within a second sequence but does not include all of the nucleotides of the second sequence.

As used herein a “nucleic acid probe or oligonucleotide” is defined as a nucleic acid capable of binding to a target nucleic acid of complementary sequence through one or more types of chemical bonds, usually through complementary base pairing, usually through hydrogen bond formation. As used herein, a probe may include natural (i.e., A, G, C, or T) or modified bases (7-deazaguanosine, inosine, etc.). In addition, the bases in a probe may be joined by a linkage other than a phosphodiester bond, so long as it does not interfere with hybridization. Thus, for example, probes may be peptide nucleic acids in which the constituent bases are joined by peptide bonds rather than phosphodiester linkages. It will be understood by one of skill in the art that probes may bind target sequences lacking complete complementarity with the probe sequence depending upon the stringency of the hybridization conditions. The probes are preferably directly labeled as with isotopes, chromophores, lumiphores, chromogens, or indirectly labeled such as with biotin to which a streptavidin complex may later bind. By assaying for the presence or absence of the probe, one can detect the presence or absence of the select sequence or subsequence.

A “labeled nucleic acid probe or oligonucleotide” is one that is bound, either covalently, through a linker or a chemical bond, or noncovalently, through ionic, van der Waals, electrostatic, or hydrogen bonds to a label such that the presence of the probe may be detected by detecting the presence of the label bound to the probe.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all.

The terms “promoter” and “expression control sequence” are used herein to refer to an array of nucleic acid control sequences that direct transcription of a nucleic acid. As used herein, a promoter includes necessary nucleic acid sequences near the start site of transcription, such as, in the case of a polymerase II type promoter, a TATA element. A promoter also optionally includes distal enhancer or repressor elements, which can be located as much as several thousand base pairs from the start site of transcription. A “constitutive” promoter is a promoter that is active under most environmental and developmental conditions. An “inducible” promoter is a promoter-that is active under environmental or developmental regulation. The term “operably linked” refers to a functional linkage between a nucleic acid expression control sequence (such as a promoter, or array of transcription factor binding sites) and a second nucleic acid sequence, wherein the expression control sequence directs transcription of the nucleic acid corresponding to the second sequence.

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

An “expression vector” is a nucleic acid construct, generated recombinantly or synthetically, with a series of specified nucleic acid elements that permit transcription of a particular nucleic acid in a host cell. The expression vector can be part of a plasmid, virus, or nucleic acid fragment. Typically, the expression vector includes a nucleic acid to be transcribed operably linked to a promoter.

The terms “identical” or percent “identity,” in the context of two or more nucleic acids or polypeptide sequences, refer to two or more sequences or subsequences that are the same or have a specified percentage of amino acid residues or nucleotides that are the same (i.e., 60% identity, preferably 65%, 70%, 75%, 80%, 85%, 90%, or 95% identity over a specified region), when compared and aligned for maximum correspondence over a comparison window, or designated region as measured using one of the following sequence comparison algorithms or by manual alignment and visual inspection. Such sequences are then said to be “substantially identical.” This definition also refers to the compliment of a test sequence. Preferably, the identity exists over a region that is at least about 25 amino acids or nucleotides in length, or more preferably over a region that is 50-100 amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. For sequence comparison of nucleic acids and proteins, the BLAST and BLAST 2.0 algorithms and the default parameters discussed below are used.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)).

An example of algorithm that is suitable for determining percent sequence identity and sequence similarity are the BLAST and BLAST 2.0 algorithms, which are described in Altschul et al., Nuc. Acids Res. 25:3389-3402 (1977) and Altschul et al., J. Mol. Biol. 215:403-410 (1990), respectively. BLAST and BLAST 2.0 are used, with the parameters described herein, to determine percent sequence identity for the nucleic acids and proteins of the invention. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always<0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) of 10, M=5, N=−4 and a comparison of both strands. For amino acid sequences, the BLASTP program uses as defaults a word length of 3, and expectation (E) of 10, and the BLOSUM62 scoring matrix (see Henikoff & Henikoff, PNAS USA 89:10915 (1989)) alignments (B) of 50, expectation (E) of 10, M=5, N=−4, and a comparison of both strands.

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

An indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

The phrase “selectively (or specifically) hybridizes to” refers to the binding, duplexing, or hybridizing of a molecule only to a particular nucleotide sequence under stringent hybridization conditions when that sequence is present in a complex mixture (e.g., total cellular or library DNA or RNA).

The phrase “stringent hybridization conditions” refers to conditions under which a probe will hybridize to its target subsequence, typically in a complex mixture of nucleic acid, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology--Hybridization with Nucleic Probes, “Overview of principles of hybridization and the strategy of nucleic acid assays” (1993). Generally, stringent conditions are selected to be about 5-10° C. lower than the thermal melting point (Tm) for the specific sequence at a defined ionic strength pH. The Tm is the temperature (under defined ionic strength, pH, and nucleic concentration) at which 50% of the probes complementary to the target hybridize to the target sequence at equilibrium (as the target sequences are present in excess, at Tm, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. For high stringency hybridization, a positive signal is at least two times background, preferably 10 times background hybridization. Exemplary high stringency or stringent hybridization conditions include: 50% formamide, 5×SSC and 1% SDS incubated at 42° C. or 5×SSC and 1% SDS incubated at 65° C., with a wash in 0.2×SSC and 0.1% SDS at 65° C. Nucleic acids that do not hybridize to each other under stringent conditions are still substantially identical if the polypeptides that they encode are substantially identical. This occurs, for example, when a copy of a nucleic acid is created using the maximum codon degeneracy permitted by the genetic code. In such cases, the nucleic acids typically hybridize under moderately stringent hybridization conditions. Exemplary “moderately stringent hybridization conditions” include a hybridization in a buffer of 40% formamide, 1 M NaCl, 1% SDS at 37° C., and a wash in 1×SSC at 45° C. A positive hybridization is at least twice background. Those of ordinary skill will readily recognize that alternative hybridization and wash conditions can be utilized to provide conditions of similar stringency. For PCR, a temperature of about 36° C. is typical for low stringency amplification, although annealing temperatures may vary between about 32° C. and 48° C. depending on primer length. For high stringency PCR amplification, a temperature of about 62° C. is typical, although high stringency annealing temperatures can range from about 50° C. to about 65° C., depending on the primer length and specificity. Typical cycle conditions for both high and low stringency amplifications include a denaturation phase of 90 ° C.-95° C. for 30 sec-2 min., an annealing phase lasting 30 sec.-2 min., and an extension phase of about 72° C. for 1-2 min.

An “analyte mixture” is any mixture that includes the target and other components. The other components are, for example, diluents, buffers, detergents, extractants, solvents, and contaminating species, debris and the like that are found mixed with the target. An analyte mixture may be, for example, a biological sample. An analyte may be, for example, a biomolecule, such as a polypeptide, a polynucleotide, a carbohydrate, or a lipid. An analyte may be an organic molecule such as a drug candidate. The analyte may be labeled with a detectable label, e.g., a fluorescent label.

A “biological sample” or a “sample” as used herein is a sample of biological tissue or fluid that contains a target component of interest. These samples are well known in the art. These samples can be tested by the methods described herein and include human and animal body fluids such as whole blood, serum, plasma, cerebrospinal fluid, urine, lymph fluids, and various external secretions of the respiratory, intestinal and genitourinary tracts, tears, saliva, milk, white blood cells, myelomas, and the like; and biological fluids such as cell extracts, cell culture supernatants; fixed tissue specimens; and fixed cell specimens. Biological samples may also include sections of tissues such as biopsy and autopsy samples or frozen sections taken for histologic purposes. Biological samples include media in which cells have been cultured. A biological sample is obtained from any living organism including viruses, prokaryotes or eukaryotes. A biological sample can be from a laboratory source or from a non-laboratory source. A biological sample may be suspended or dissolved in liquid materials such as buffers, extractants, solvents and the like.

A “locking agent” is any agent used to reduce nonspecific binding, e.g., of proteins such as antibodies, antigens, ligands, receptors; nucleic acids, such as DNA or RNA, without interfering with the studied proteins or the detection of the desired molecules. A preferred blocking agent is a macromolecule, large enough to establish a stable attachment to a surface and small enough to move easily among components of an analyte mixture.

“Antibody” refers to a polypeptide comprising a framework region from an immunoglobulin gene or fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (VL) and variable heavy chain (VH) refer to these light and heavy chains respectively.

Antibodies exist, e.g., as intact immunoglobulins or as a number of well-characterized fragments produced by digestion with various peptidases. Thus, for example, pepsin digests an antibody below the disulfide linkages in the hinge region to produce F(ab)′2, a dimer of Fab which itself is a light chain joined to VH-CH1 by a disulfide bond. The F(ab)′2 may be reduced under mild conditions to break the disulfide linkage in the hinge region, thereby converting the F(ab)′2 dimer into an Fab′ monomer. The Fab′ monomer is essentially Fab with part of the hinge region (see Fundamental Immunology (Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990)).

For preparation of monoclonal or polyclonal antibodies, any technique known in the art can be used (see, e.g., Kohler & Milstein, Nature 256:495-497 (1975); Kozbor et al., Immunology Today 4: 72 (1983); Cole et al., pp. 77-96 in Monoclonal Antibodies and Cancer Therapy, Alan R. Liss, Inc. (1985)). Techniques for the production of single chain antibodies (U.S. Pat. No. 4,946,778) can be adapted to produce antibodies to polypeptides of this invention. Also, transgenic mice, or other organisms such as other mammals, may be used to express humanized antibodies. Alternatively, phage display technology can be used to identify antibodies and heteromeric Fab fragments that specifically bind to selected antigens (see, e.g., McCafferty et al., Nature 348:552-554 (1990); Marks et al., Biotechnology 10:779-783 (1992)).

A “chimeric antibody” is an antibody molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity.

The term “immunoassay” is an assay that uses an antibody to specifically bind an antigen. The immunoassay is characterized by the use of specific binding properties of a particular antibody to isolate, target, detect, and/or quantify the antigen.

The phrase “specifically (or selectively) binds” to an antibody or “specifically (or selectively) immunoreactive with,” when referring to a protein or peptide, refers to a binding reaction that is determinative of the presence of the protein in a heterogeneous population of proteins and other biologics. Thus, under designated immunoassay conditions, the specified antibodies bind to a particular protein at least two times the background and do not substantially bind in a significant amount to other proteins present in the sample. Specific binding to an antibody under such conditions may require an antibody that is selected for its specificity for a particular protein. For example, polyclonal antibodies raised to a particular protein can be selected to obtain only those polyclonal antibodies that are specifically immunoreactive with family members of the particular protein and not with other proteins. This selection may be achieved by subtracting out antibodies that cross-react with molecules from other species. In addition, polyclonal antibodies raised to polymorphic variants, alleles, orthologs, and conservatively modified variants of the particular protein can be selected to obtain only those antibodies that recognize specific fragments of the particular protein. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Antibodies, A Laboratory Manual (1988) for a description of immunoassay formats and conditions that can be used to determine specific immunoreactivity). Typically a specific or selective reaction will be at least twice background signal or noise and more typically more than 10 to 100 times background.

The phrase “selectively associates with” refers to the ability of a nucleic acid to “selectively hybridize” with another as defined above, or the ability of an antibody to “selectively (or specifically) bind to a protein, as defined above.

By “host cell” is meant a cell that contains an expression vector and supports the replication or expression of the expression vector. Host cells may be prokaryotic cells such as E. coli or eukaryotic cells such as yeast, insect, amphibian, or mammalian cells such as CHO, HeLa and the like, e.g., cultured cells, explants, and cells in vivo.

III. Detecting Proliferation of Cells

In one embodiment, the present invention provides method for determining the relative prevalence of one or more cell types in a mixture of, e.g., 2, 4, 6, 8, 10, 12, 20, 40, 60, 80, or 100 or more cell types in a culture. For example, the methods are useful for detecting proliferation of one or more of the cell types, and for detecting induction of cell death of one or more of the cell types. Each cell type contains a nucleic acid comprising a unique polynucleotide tag. Each polynucleotide tag comprises a first primer binding site on a first strand and a second primer binding site on a second strand. The second strand is complementary to the first strand. Each polynucleotide tag further comprises a unique restriction site at a preselected position relative to the first primer binding site. The position of the unique restriction site in each polynucleotide tag differs from the position of the unique restriction site in every other polynucleotide tag. In some embodiments, the cells are incubated in a medium and under conditions suitable for proliferation of the cells.

The relative prevalence of each of the cell types is detected by (1) amplifying the polynucleotide tags with a pair of primers that hybridize to the first and second primer binding sites, thereby producing amplified products that correspond to the polynucleotide tags that are present; (2) cleaving the amplified products with a restriction enzyme that cleaves the amplified products at unique restriction sites (i.e., each amplified polynucleotide tag is cleaved at a site located at a different position relative to the first primer binding site), thereby forming cleaved amplification products; and (3) detecting the cleaved amplification products. For example, the amount of a first cleaved amplification product is correlated with the relative prevalence of the first cell type and the amount of a second cleaved amplification product is correlated with the relative prevalence of the second cell type.

Suitable cells include any prokaryotic and eukaryotic cell, including, for example, yeast cells, bacterial cells, and mammalian cells. In an exemplary embodiments, the cells are recombinant yeast cells, i.e., yeast two hybrids that express two interacting proteins (a bait protein and a prey protein) and cell proliferation or cell death is dependent upon the interaction between the two proteins (see, e.g., Coates and Hall, J. Pathol. 199(1):4-7 (2003); Gietz et al., Methods Mol.Biol. 185:471-86 (2002); Ma, Prog. Drug Res. 57:117-62 (2001); Nagpal et al., Methods Mol. Biol. 176:359-76 (2001); Le Douarin et al., Methods Mol. Biol. 176:227-48 (2001); Serebriiskii, et al., Methods Mol. Biol. 175:415-54 (2001)). or recombinant bacterial cells, i.e., bacterial two hybrids (see, e.g., Joung, J. Cell Biochem. Suppl. Suppl 37:53-7 (2001)).

This invention relies on routine techniques in the field of recombinant genetics. Generally, the nomenclature and the laboratory procedures in recombinant DNA technology described below are those well known and commonly employed in the art. Standard techniques are used for cloning, DNA and RNA isolation, amplification and purification. Generally enzymatic reactions involving DNA ligase, DNA polymerase, restriction endonucleases and the like are performed according to the manufacturer's specifications. Basic texts disclosing the general methods of use in this invention include Sambrook et al. 2001, supra; Kriegler, Gene Transfer and Expression: A Laboratory Manual (1990); and Current Protocols in Molecular Biology (Ausubel et al., eds., 1994)).

For nucleic acids, sizes are given in either kilobases (kb) or base pairs (bp). These are estimates derived from agarose or acrylamide gel electrophoresis, from sequenced nucleic acids, or from published DNA sequences. For proteins, sizes are given in kilodaltons (kDa) or amino acid residue numbers. Proteins sizes are estimated from gel electrophoresis, from sequenced proteins, from derived amino acid sequences, or from published protein sequences.

Oligonucleotides that are not commercially available can be chemically synthesized according to the solid phase phosphoramidite triester method first described by Beaucage & Caruthers, Tetrahedron Letts. 22:1859-1862 (1981), using an automated synthesizer, as described in Van Devanter et. al., Nucleic Acids Res. 12:6159-6168 (1984). Purification of oligonucleotides is by either native acrylamide gel electrophoresis or by anion-exchange HPLC as described in Pearson & Reanier, J. Chrom. 255:137-149 (1983).

A. Culture of Cells

This invention relies upon routine techniques in the field of cell culture. Suitable cell culture methods and conditions can be determined by those of skill in the art using known methodology (see, e.g., Sambrook and Russell, MOLECULAR CLONING: A LABORATORY MANUAL (3rd ed. 2001) and Freshney et al., CULTURE OF ANIMAL CELLS (3rd ed. 1994)). In general, the cell culture environment includes consideration of such factors as the substrate for cell growth, cell density and cell contract, the gas phase, the medium, and temperature. Suitable cells for use in the methods of the invention include any prokaryotic and eukaryotic cells including, for example, yeast, bacteria, and mammalian cells.

In an exemplary embodiment, the cells are recombinant yeast cells. In some cases, the yeast cells are yeast two hybrid cells expressing two interacting fusion proteins that drive cell proliferation (see, e.g., Current Protocols in Molecular Biology Volumes 1-4, John Wiley & Sons, Inc. (Ausubel et al., eds., 1994-1999); Causier et al., Plant Mol. Biol. 50(6):855-70 (2002); Coates and Hall, J. Pathol. 199(1):4-7 (2003); and Ma, Prog. Drug Res. 57:117-62 (2001)) One of the fusion proteins comprises a DNA binding domain and the other fusion protein comprises a transcription activation domain that interacts with the DNA binding domain. When the two fusion proteins interact, the DNA binding domain and the transcription activation domain interact to drive transcription of an essential gene, e.g. of HIS3. In some cases, the yeast cells are yeast two hybrid cells expressing bait and prey proteins, wherein the bait protein is a known member of a pair of proteins that interact and the prey protein is protein that may interact with the bait protein. If the prey protein and the bait protein interact, cell proliferation proceeds and an amplified product will be produced and can be detected as described.

Incubation is generally performed under conditions known to be optimal for cell growth. For yeast cells, such conditions may include for example a temperature of approximately 30° C. and agitation. The duration of the incubation can vary widely, depending on the desired results. Proliferation is conveniently determined by measuring the optical density of the cell culture at 600 nm (OD₆₀₀). For mammalian cells, such conditions may include for example a temperature of approximately 37° C. and a humidified atmosphere containing approximately 5% CO₂. The duration of the incubation can vary widely, depending on the desired results. Proliferation is conveniently determined using ³H thymidine incorporation or BrdU labeling.

Plastic (e.g., polystyrene) or glass dishes, flasks, roller bottles, or microcarriers in suspension may be used to culture cells according to the methods of the present invention. Suitable culture vessels include, for example, multi-well plates (e.g., 6, 12, 24, 28, 96, 384, or 1536 well microtiter plate), petri dishes, tissue culture tubes, flasks, roller bottles, and the like.

Cells are grown at optimal densities that are determined empirically based on the cell type. Cells are passaged when the cell density is above optimal.

Cultured cells are normally grown in an incubator that provides a suitable temperature, e.g., the body temperature of the animal from which is the cells were obtained, accounting for regional variations in temperature. Generally, 30° C. is the preferred temperature for yeast cell culture. Generally, 37° C. is the preferred temperature for mammalian cell culture. Most incubators are humidified to approximately atmospheric conditions for mammalian cell culture.

Important constituents of the gas phase are oxygen and carbon dioxide. Typically, atmospheric oxygen tensions are used for mammalian cell cultures. Culture vessels are usually vented into the incubator atmosphere to allow gas exchange by using gas permeable caps or by preventing sealing of the culture vessels. Carbon dioxide plays a role in pH stabilization, along with buffer in the cell media and is typically present at a concentration of 1-10% in the incubator. The preferred CO₂ concentration typically is 5%.

Defined cell media are available as packaged, premixed powders or presterilized solutions. Examples of commonly used media for yeast cells include CM, SC, Drop-out Media, SMM, SD, and YPD (see, e.g., Sambrook, 2001, supra). Yeast cell culture media are often supplemented with amino acids. Examples of commonly used media for mammalian cells include DME, RPMI 1640, DMEM, Iscove's complete media, or McCoy's Medium (see, e.g., GibcoBRL/Life Technologies Catalogue and Reference Guide; Sigma Catalogue). Defined cell culture media are often supplemented with 5-20% serum, typically heat inactivated serum, e.g., human, horse, calf, and fetal bovine serum. The culture medium is usually buffered to maintain the cells at a pH preferably from 7.2-7.4. Other supplements to the media include, e.g., antibiotics, amino acids, sugars, and growth factors.

B. Transfection

Standard transfection methods are used to produce bacterial, mammalian, yeast or insect cell lines or types that contain the unique oligonucleotide tags described herein. Transformation of eukaryotic and prokaryotic cells are performed according to standard techniques (see, e.g., Morrison, J. Bact. 132:349-351 (1977); Clark-Curtiss & Curtiss, Methods in Enzymology 101:347-362 (Wu et al., eds, 1983).

Any of the well-known procedures for introducing foreign nucleotide sequences into host cells may be used. These include the use of calcium phosphate transfection, polybrene, protoplast fusion, electroporation, biolistics, liposomes, microinjection, plasma vectors, viral vectors and any of the other well known methods for introducing cloned genomic DNA, cDNA, synthetic DNA or other foreign genetic material into a host cell (see, e.g., Sambrook et al., supra). It is only necessary that the particular genetic engineering procedure used be capable of successfully introducing the oligonucleotide tag into the host cell.

C. Amplification/Cleavage

A general overview of the applicable amplification technology can be found in PCR Protocols: A Guide to Methods and Applications (Innis et al. eds. (1990)) and PCR Technology: Principles and Applications for DNA Amplification (Erlich, ed. (1992)).

PCR permits the copying, and resultant amplification of a target nucleic acid, e.g., an oligonucleotide tag. Briefly, a target nucleic acid, e.g. genomic DNA comprising an oligonucleotide tag and isolated from a cell culture or a cell lysate, is combined with a sense and antisense primers, dNTPs, DNA polymerase and other reaction components. (See, Innis et al., supra) The sense primer can anneal to the antisense strand of a DNA sequence of interest, i.e., a sequence complementary to the oligonucleotide tag. The antisense primer can anneal to the sense strand of the DNA sequence, downstream of the location where the sense primer anneals to the DNA target. In the first round of amplification, the DNA polymerase extends the antisense and sense primers that are annealed to the target nucleic acid. The first strands are synthesized as long strands of indiscriminate length. In the second round of amplification, the antisense and sense primers anneal to the parent target nucleic acid and to the complementary sequences on the long strands. The DNA polymerase then extends the annealed primers to form strands of discrete length that are complementary to each other. The subsequent rounds serve to predominantly amplify the DNA molecules of the discrete length.

In general, PCR and other methods of amplification use primers which anneal to either end of the DNA of interest. For example, the oligonucleotide tags may be amplified using isolated nucleic acid primer pairs having the following sequences: 5′ primer: GAAGTTATCGGAGGAATTGGCTCGAGG (SEQ ID NO:3) and 3′ primer AGGAGAGGGTTAGGGATAGGCTTACCG (SEQ ID NO:4).

In some embodiments of the invention, amplification of the oligonucleotide tag is performed using a rolling circle amplification as described in, e.g., U.S. Pat. Nos. 5,854,033 and 6,210,884. Briefly, a rolling circle replication primer hybridizes to circular open circle probes (OCP) molecules followed by rolling circle replication of the OCP using a strand-displacing DNA polymerase. Amplification takes place during rolling circle replication in a single reaction cycle and results in large DNA molecules containing tandem repeats of the OCP sequence (TS-DNA). The TS-DNA molecules generated by RCA are of high molecular weight and low complexity; the complexity being the length of the open circle probe. RCA is easily performed in a multiplex assay format by using different oligonucleotide tags as open circle probes.

Target DNA sequences may be isolated using a variety of techniques. For example, methods are known for lysing organisms and preparing extracts or purifying DNA. See, Current Protocols in Molecular Biology Volumes 1-4, John Wiley & Sons, Inc. (Ausubel et al., eds., 1994-1999).

D. Reaction Components
1. Oligonucleotides

The oligonucleotides that are used in the present invention as well as oligonucleotides designed to detect amplification products can be chemically synthesized, as described above. These oligonucleotides can be labeled with radioisotopes, chemiluminescent moieties, or fluorescent moieties. Such labels are useful for the characterization and detection of amplification products using the methods and compositions of the present invention.

The primer components may be present in the PCR reaction mixture at a concentration of, e.g., between 0.1 and 1.0 μM. The concentration of the target primers can be from about 0.1 to about 0.75 μM. The primer length can be between, e.g., 15-100 nucleotides in length and preferably have 40-60% G and C composition. In the choice of primer, it is preferable to have exactly matching bases at the 3′ end of the primer but this requirement decreases to relatively insignificance at the 5′ end. Preferably, the primers of the invention all have approximately the same melting temperature.
2. Buffer

Buffers that may be employed are borate, phosphate, carbonate, barbital, Tris, etc. based buffers. (See, U.S. Pat. No. 5,508,178). The pH of the reaction should be maintained in the range of about 4.5 to about 9.5. (See, U.S. Pat. No. 5,508,178. The standard buffer used in amplification reactions is a Tris based buffer between 10 and 50 mM with a pH of around 8.3 to 8.8. (See Innis et al., supra.).

One of skill in the art will recognize that buffer conditions should be designed to allow for the function of all reactions of interest. Thus, buffer conditions can be designed to support the amplification reaction as well as any subsequent restriction enzyme reactions. A particular reaction buffer can be tested for its ability to support various reactions by testing the reactions both individually and in combination.
3. Salt Concentration

The concentration of salt present in the reaction can affect the ability of primers to anneal to the target nucleic acid. (See, Innis et al.). Potassium chloride is added up to a concentration of about 50 mM to the reaction mixture to promote primer annealing. Sodium chloride can also be added to promote primer annealing. (See, Innis et al.).
4. Magnesium Ion Concentration

The concentration of magnesium ion in the reaction can affect amplification of the target sequence(s). (See, Innis et al.). Primer annealing, strand denaturation, amplification specificity, primer-dimer formation, and enzyme activity are all examples of parameters that are affected by magnesium concentration. (See, Innis et al.). Amplification reactions should contain about a 0.5 to 2.5 mM magnesium concentration excess over the concentration of dNTPs. The presence of magnesium chelators in the reaction can affect the optimal magnesium concentration. A series of amplification reactions can be carried out over a range of magnesium concentrations to determine the optimal magnesium concentration. The optimal magnesium concentration can vary depending on the nature of the target nucleic acid(s) and the primers being used, among other parameters.
5. Deoxynucleotide Triphosphate Concentration

Deoxynucleotide triphosphates (dNTPs) are added to the reaction to a final concentration of about 20 μM to about 300 μM. Typically, each of the four dNTPs (G, A, C, T) are present at equivalent concentrations. (See, Innis et al.). In some embodiments, the dNTP's are labeled, e.g., with dye, isotopes, and the like.
6. Nucleic Acid Polymerase

A variety of DNA dependent polymerases are commercially available that will function using the methods and compositions of the present invention. For example, Taq DNA Polymerase may be used to amplify target DNA sequences. The PCR assay may be carried out using as an enzyme component a source of thermostable DNA polymerase suitably comprising Taq DNA polymerase which may be the native enzyme purified from Thermus aquaticus and/or a genetically engineered form of the enzyme. Other commercially available polymerase enzymes include, e.g., Taq polymerases marketed by Promega or Pharmacia. Other examples of thermostable DNA polymerases that could be used in the invention include DNA polymerases obtained from, e.g., Thermus and Pyrococcus species. Concentration ranges of the polymerase may range from 1-5 units per reaction mixture. The reaction mixture is typically between 20 and 100 μl.

In some embodiments, a “hot start” polymerase can be used to prevent extension of mispriming events as the temperature of a reaction initially increases. Hot start polymerases can have, for example, heat labile adducts requiring a heat activation step (typically 95° C. for approximately 10-15 minutes) or can have an antibody associated with the polymerase to prevent activation.

In some embodiments, rolling circle DNA polymerases are used. Such DNA polymerases perform rolling circle replication of primed single-stranded circles (see, e.g., U.S. Pat. No. 6,210,884. For rolling circle replication, it is preferred that a DNA polymerase be capable of displacing the strand complementary to the template strand, termed strand displacement, and lack a 5′ to 3′ exonuclease activity. Strand displacement is necessary to result in synthesis of multiple tandem copies of the ligated OCP. A 5′ to 3′ exonuclease activity, if present, might result in the destruction of the synthesized strand. It is also preferred that DNA polymerases for use in the disclosed method are highly processive. The suitability of a DNA polymerase for use in the disclosed method can be readily determined by assessing its ability to carry out rolling circle replication. Preferred rolling circle DNA polymerases are bacteriophage Φ29 DNA polymerase (U.S. Pat. Nos. 5,198,543 and 5,001,050), phage M2 DNA polymerase (Matsumoto et al., Gene 84:247 (1989)), phage Φ PRD1 DNA polymerase (Jung et al., PNAS USA 84:8287 (1987)), VENT™. DNA polymerase (Kong et al., J. Biol. Chem. 268:1965-1975 (1993)), Klenow fragment of DNA polymerase I (Jacobsen et al., Eur. J. Biochem. 45:623-627 (1974)), T5 DNA polymerase (Chatterjee et al., Gene 97:13-19 (1991)), PRD1 DNA polymerase (Zhu and Ito, Biochim. Biophys. Acta. 1219:267-276 (1994)), and T4 DNA polymerase holoenzyme (Kaboord and Benkovic, Curr. Biol. 5:149-157 (1995)).

Strand displacement can be facilitated through the use of a strand displacement factor, such as helicase. It is considered that any DNA polymerase that can perform rolling circle replication in the presence of a strand displacement factor is suitable for use in the disclosed method, even if the DNA polymerase does not perform rolling circle replication in the absence of such a factor. Strand displacement factors useful in RCA include BMRF1 polymerase accessory subunit (Tsurumi et al., J. Virology 67(12):7648-7653 (1993)), adenovirus DNA-binding protein (Zijderveld and van der Vliet, J. Virology 68(2):1158-1164 (1994)), herpes simplex viral protein ICP8 (Boehmer and Lehman, J. Virology 67(2):711-715 (1993); Skaliter and Lehman, PNAS USA 91(22):10665-10669 (1994)), single-stranded DNA binding proteins (SSB; Rigler and Romano, J. Biol. Chem. 270:8910-8919 (1995)), and calf thymus helicase (Siegel et al., J. Biol. Chem. 267:13629-13635 (1992)).

The ability of a polymerase to carry out rolling circle replication can be determined by using the polymerase in a rolling circle replication assay such as those described in Fire and Xu, PNAS USA 92:4641-4645 (1995).
7. Other Agents

Additional agents are sometime added to the reaction to achieve the desired results. For example, DMSO can be added to the reaction, but is reported to inhibit the activity of Taq DNA Polymerase. Nevertheless, DMSO has been recommended for the amplification of multiple target sequences in the same reaction. (See, Innis et al. supra). Stabilizing agents such as gelatin, bovine serum albumin, and non-ionic detergents (e.g. Tween-20) are commonly added to amplification reactions. (See, Innis et al. supra).

E. Amplification and Cleavage

Amplification of an RNA or DNA template using reactions is well known (see, U.S. Pat. Nos. 4,683,195 and 4,683,202; PCR Protocols: A Guide to Methods and Applications (Innis et al., eds, 1990)). Methods such as polymerase chain reaction (PCR) and ligase chain reaction (LCR) can be used to amplify nucleic acid sequences of target DNA sequences (e.g., oligonucleotide tags). The reaction is preferably carried out in a thermal cycler to facilitate incubation times at desired temperatures.

Exemplary PCR reaction conditions typically comprise either two or three step cycles. Two step cycles have a denaturation step followed by a hybridization/elongation step. Three step cycles comprise a denaturation step followed by a hybridization step followed by a separate elongation step.

Exemplary rolling circle amplification reactions typically comprise a continuous isothermal replication step.

Amplified PCR products can be cleaved by any restriction enzymes known in the art, including, for example, Aat II, Acc65 I, Acc I, Aci I, Acl I, Afe I, Afl II, Afl III, Age I, Ahd I, Ale I, Alu I, Alw I, AlwN I, Apa I, ApaL I, Apo I, Asc I, Ase I, AsiS I, Ava I, Ava II, Avr II, Bae I, BamH I, Ban I, Ban II, Bbs I, Bbv I, BbvC I, Bcc I, BceA I, Bcg I, BciV I, Bcl I, Bfa I, BfrB I, BfuA I, BfuC I, Bgl I, Bgl II, Blp I, Bme1580 I, BmgB I, Bmr I, Bmt I, Bpm I, Bpu10 I, BpuE I, BsaA I, BsaB I, BsaH I, Bsa I, BsaJ I, BsaW I, BsaX I, BseR I, BseY I, Bsg I, BsiE I, BsiHKA I, BsiW I, Bsl I, BsmA, I, BsmB I, BsmF I, Bsm I, BsoB I, Bsp1286 I, BspCN I, BspD I, BspE I, BspH I, BspM I, BsrB I, BsrD I, BsrF I, BsrG I, Bsr I, BssH II, BssK I, BssS I, BstAP I, BstB I, BstE II, BstF5 I, BstN I, BstU I, BstX I, BstY I, BstZ17 I, Bsu36 I, Btg I, Bts I, Cac8 I, Cla I, Dde I, Dpn I, Dpn II, Dra I, Dra III, Drd I, Eae I, Eag I, Ear I, Eci I, EcoN I, EcoO109 I, EcoR I, EcoR V, Fat I, Fau I, Fnu4H I, Fok I, Fse I, Fsp I, Hae II, Hae III, Hga I, Hha I, Hinc II, Hind III, Hinf I, HinPI I, Hpa I, Hpa II, Hpyl 88 I, Hpy 188 III, Hpy99 I, HpyCH4III, HpyCH4IV, HpyCH4V, Hph I, Kas I, Kpn I, Mbo I, Mbo II, Mfe I, Mlu I, Mly I, Mme I, Mn1 I, Msc I, Mse I, Msl I, MspAl I, Msp I, Mwo I, Nae I, Nar I, Nci I, Nco I, Nde I, NgoM IV, Nhe I, Nla III, Nla IV, Not I, Nru I, Nsi I, Nsp I, Pac I, PaeR7 I, Pci I, PflF I, PflM I, Pho I, Ple I, Pme I, Pml I, PpuM I, PshA I, Psi I, PspG I, PspOM I, Pst I, Pvu I, Pvu II, Rsa I, Rsr II, Sac I, Sac II, Sal I, Sap I, Sau3A, Sau96 I, Sbf I, Sca I, ScrF I, SexA I, SfaN I, Sfc I, Sfi I, Sfo I, SgrA I, Sma I, Sm1 I, SnaB I, Spe I, Sph I, Ssp I, Stu I, Sty I, StyD4 I, Swa I, Taqa I, Tfi I, Tli I, Tse I, Tsp45 I, Tsp509 I, TspR I, Tth111 I, Xba I, Xcm I, Xho I, Xma I, Xmn I, Zra I. Specific reaction conditions for cleavage of the amplified products with restriction enzymes are well known to those of skill in the art.

The cleaved product can be detected by any means known in the art including, for example, by electrophoresis of the amplified, cleaved product followed by detection of the product. Electrophoresis includes, e.g., gel electrophoresis, capillary electrophoresis, polyacrylamide gel electrophoresis, and the like. The amount of cleaved amplification product is correlated with the proliferation of the cell type which contained the particular oligonucleotide tag which was amplified.

F. Screening Libraries of Compounds

In certain embodiments, the invention provides methods of screening a library of compounds to identify those compounds that can affect the relative prevalence of different cell types in a mixture of cell types. For example, the invention provides methods of screening libraries of compounds to identify those compounds that modulate cell proliferation, or that cause cell death. These methods involve providing mixtures of cell types, each type containing a unique polynucleotide tag, contacting the cell mixture with a member of a library of compounds, and incubating the cells under conditions suitable for, for example, proliferation of the cells. The relative prevalence of one or more of the different cell types is detected as described above, i.e., by amplification and detection of the oligonucleotide tags. In some embodiments, only oligonucleotide tags from cell types that proliferate in the presence of the library member will be amplified. In other embodiments, oligonucleotide tags from cell types whose proliferation is partially inhibited or retarded in the presence of the library member will be amplified.

In an exemplary embodiment, the cells are recombinant cells. For example, the cells can be recombinant yeast, mammalian, plant, or cells, and the like. In some embodiments, the cells contain “two hybrid” systems in which two interacting proteins are expressed, and cell proliferation is dependent on the interaction between the two proteins. For example, two hybrid cells can express a pair of fusion proteins that interact to drive transcription of an essential gene. One fusion protein comprises a DNA binding domain and the other fusion protein comprises an activation domain from a transcription factor. The cell also contains a response element to which the DNA binding domain can bind; the response element is operably linked to the essential gene. If the fusion proteins interact, the DNA binding domain and the activation domain act in concert to drive transcription of the essential gene. In some embodiments, both of the interacting proteins are known. In other embodiments, only one of the interacting proteins is known, i.e., bait and prey proteins. Compounds that, for example, restore, inhibit, retard, or enhance the ability of the cells to proliferate are identified as compounds that modulate cell proliferation.

Conventionally, new chemical entities with useful properties are generated by identifying a chemical compound (called a “lead compound”) with some desirable property or activity, e.g., inhibiting activity, creating variants of the lead compound, and evaluating the property and activity of those variant compounds, i.e., their ability to restore, inhibit, retard, or enhance the ability of the cells to proliferate. Often, high throughput screening (HTS) methods are employed for such an analysis.

In one preferred embodiment, high throughput screening methods involve providing a library containing a large number of potential therapeutic compounds (candidate compounds). Such “combinatorial chemical libraries” are then screened in one or more assays to identify those library members (particular chemical species or subclasses) that display a desired characteristic activity, i.e., modulation of cell proliferation. Each library member is contacted with a mixture of cell types, each cell type containing a unique oligonucleotide tag that can be amplified and detected if the cell grows after contact with that library member. In some embodiments, the cells are in an array format, e.g., in a microtiter plate. In some embodiments, the cells are yeast two hybrid cells which require the interaction of two proteins to proliferate. Compounds that affect the interaction of the two proteins can modulate the proliferation of the cells and are also identified as modulators of cell proliferation. Compounds identified using high throughput screening methods can serve as conventional “lead compounds” or can themselves be used as potential or actual therapeutics.

A combinatorial chemical library is a collection of diverse chemical compounds generated by either chemical synthesis or biological synthesis by combining a number of chemical “building blocks” such as reagents. For example, a linear combinatorial chemical library, such as a polypeptide (e.g., mutein) library, is formed by combining a set of chemical building blocks called amino acids in every possible way for a given compound length (i.e., the number of amino acids in a polypeptide compound). Millions of chemical compounds can be synthesized through such combinatorial mixing of chemical building blocks (Gallop et al, J. Med. Chem. 37(9):1233-1251 (1994)).

Preparation and screening of combinatorial chemical libraries is well known to those of skill in the art. Such combinatorial chemical libraries include, but are not limited to, peptide libraries (see, e.g., U.S. Pat. No. 5,010,175, Furka, Pept. Prot. Res. 37:487-493 (1991), Houghton et al., Nature, 354:84-88 (1991)), peptoids (PCT Publication No WO 91/19735), encoded peptides (PCT Publication WO 93/20242), random bio-oligomers (PCT Publication WO 92/00091), benzodiazepines (U.S. Pat. No. 5,288,514), diversomers such as hydantoins, benzodiazepines and dipeptides (Hobbs et al., Proc. Nat. Acad. Sci. USA 90:6909-6913 (1993)), vinylogous polypeptides (Hagihara et al., J. Amer. Chem. Soc. 114:6568 (1992)), nonpeptidal peptidomimetics with a Beta-D-Glucose scaffolding (Hirschmann et al., J. Amer. Chem. Soc. 114:9217-9218 (1992)), analogous organic syntheses of small compound libraries (Chen et al., J. Amer. Chem. Soc. 116:2661 (1994)), oligocarbamates (Cho, et al., Science 261:1303 (1993)), and/or peptidyl phosphonates (Campbell et al., J. Org. Chem. 59:658 (1994)). See, generally, Gordon et al., J. Med. Chem. 37:1385 (1994), carbohydrate libraries (see, e.g., Liang et al., Science 274:1520-1522 (1996), and U.S. Pat. No. 5,593,853), and small organic molecule libraries (see, e.g., benzodiazepines, Baum, C&EN, January 18, page 33 (1993); isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, U.S. Pat. No. 5,288,514, compounds that regulate adenyl cyclase and cyclic AMP, such as, for example, forskolin and its derivatives, U.S. Pat. Nos. 5,789,439; 5,350,864, and 4,954,642.

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.).

A number of well known robotic systems have also been developed for solution phase chemistries. These systems include automated workstations like the automated synthesis apparatus developed by Takeda Chemical Industries, LTD. (Osaka, Japan) and many robotic systems utilizing robotic arms (Zymate II, Zymark Corporation, Hopkinton, Mass.; Orca, Hewlett-Packard, Palo Alto, Calif.), which mimic the manual synthetic operations performed by a chemist. The above devices, with appropriate modification, are suitable for use with the present invention. In addition, numerous combinatorial libraries are themselves commercially available (see, e.g., ComGenex, Princeton, N.J., Asinex, Moscow, Ru, Tripos, Inc., St. Louis, Mo., ChemStar, Ltd, Moscow, RU, 3D Pharmaceuticals, Exton, Pa., Martek Biosciences, Columbia, Md., etc.).

The assays to identify compounds that modulate cellular proliferation are amenable to high throughput screening. High throughput assays for evaluating the presence, absence, quantification, or other properties of particular nucleic acids or protein products are well known to those of skill in the art. Similarly, binding assays and reporter gene assays are similarly well known. Thus, e.g., U.S. Pat. No. 5,559,410 discloses high throughput screening methods for proteins, U.S. Pat. No. 5,585,639 discloses high throughput screening methods for nucleic acid binding (i.e., in arrays), while U.S. Pat. Nos. 5,576,220 and 5,541,061 disclose high throughput methods of screening for ligand/antibody binding.

Robotic systems for high throughput screening are described in, for example, U.S. Patent Publication 2002-0090320 A1, and are commercially available from Kalypsys, Inc. Other high throughput screening systems also are commercially available (see, e.g., Zymark Corp., Hopkinton, Mass.; Air Technical Industries, Mentor, Ohio; Beckman Instruments, Inc. Fullerton, Calif.; Precision Systems, Inc., Natick, Mass., etc.). These systems typically automate procedures, including sample and reagent pipeting, liquid dispensing, timed incubations, and final readings of the microplate in detector(s) appropriate for the assay. These configurable systems provide high throughput and rapid start up as well as a high degree of flexibility and customization. The manufacturers of such systems provide detailed protocols for various high throughput systems. Thus, e.g., Zymark Corp. provides technical bulletins describing screening systems for detecting the modulation of gene transcription, ligand binding, and the like.

IV. Detecting Targets in a Sample

Yet another embodiment of the present invention provides a method for detecting the presence or absence of targets in a sample, e.g., an aqueous sample comprising an analyte mixture. Affinity interaction assays are used to detect specific binding interactions between binding moieties and their targets, e.g., antibody-antigen interactions, nucleic acid-protein interactions, and receptor ligand interactions. The binding moieties comprise unique polynucleotide tags. Targets that can be detected include, for example, polypeptides (e.g., antigens, receptors, and ligands) and nucleic acids (e.g., DNA and RNA). In the methods of the invention, binding moieties that specifically interact with, i.e., bind to, their target are detected by amplification and detection of the unique polynucleotide tag associated with the binding moiety. Affinity interaction assays that comprise detection of an oligonucleotide tag by amplification of the tag are described in, e.g., Schweitzer et al., PNAS USA 97(18):10113-10119 (2000) and Hendrickson et al., Nucl. Acids Res. 23(3):522-529 (1995)), and Joerger et al., Clin. Chem. 41(9):1371-1377 (1995) which describe the use of unique DNA labels which differ in size.

A sample (i.e. an analyte mixture) comprising at least one target or suspected of comprising at least one target is contacted with a detection reagent comprising a binding moiety and a unique polynucleotide tag as described in detail above. If the target is present in the analyte mixture, a binding moiety-target complex is formed. The presence of the target in the sample can be detected by amplification of the oligonucleotide tag followed by cleavage and detection of the amplified product as described in detail above.

One of skill in the art will appreciate that the binding moiety and the unique polynucleotide tag can conveniently be linked using any methods known in the art. For example, preparation of protein-nucleic acid conjugates is described in, e.g., Schweitzer et al., supra and Hendrickson et al., supra). Oligonucleotides comprising a binding moiety and a polynucleotide tag can be synthesized as described above.

In some embodiments, the target or mixture of targets is bound to or immobilized on a surface, e.g., in an array format (see, e.g., Schweitzer et al., supra and U.S. Pat. Nos. 6,534,307; 6,534,270; 6,511,849; 6,500,620; 6,423,552; 6,110,426; and 5,807,522). The bound or immobilized target specifically interacts with the binding moiety linked to an oligonucleotide tag to form a binding moiety-target complex. The presence of the target in the sample can be detected by amplification of the oligonucleotide tag followed by cleavage and detection of the amplified product as described in detail above. One of skill in the art will appreciate that the target may directly contact a surface or may be linked to the surface via a second molecule, e.g., an immunoglobulin, biotin, or another linker known in the art.

Antigen-antibody binding assays are well known in the art and are described in, e.g., Harlow and Lane, supra., Schweitzer et al., supra, Hendrickson et al., supra, and Joerger et al., supra. Receptor-ligand binding assays are also well known in the art (see, e.g., Fernandes, Curr. Opin. Chem. Biol. 2(5):597-603 (1998); Goldsmith, Semin. Nucl. Med. 27(2):85-93 (1997); Shaw, and Curr. Opin. Biotechnol. 3(1):55-8 (1992)). Likewise, RNA-protein and DNA-protein binding assays have been previously described (see, e.g., Tuerk and Gold, Science 249: 505 (1990); Irvine et al., J. Mol. Biol. 222: 739 (1991); Oliphant et al., Mol. Cell Biol. 9: 2944 (1989); Blackwell et al., Science 250: 1104 (1990); Pollock and Treisman, Nuc. Acids Res. 18: 6197 (1990); Thiesen and Bach, Nuc. Acids Res. 18: 3203 (1990); Bartel et al., Cell 57: 529 (1991); Stormo and Yoshioka, PNAS 88: 5699 (1991); and Bock et al., Nature 355: 564 (1992)), small molecule binding finctions (Ellington and Szostak, Nature 346: 818 (1990); Ellington and Szostak, Nature 355: 850 (1992)).

The target is optionally tagged with a detectable label, or may be detected using a detectably labeled secondary antibody or binding reagent. Detectable labels or tags are known in the art, and include fluorescent, calorimetric and radiolabels, for instance. The particular label or detectable group used in the assay is not a critical aspect of the invention, as long as it does not significantly interfere with the specific binding of targets in the analyte mixture to the binding moiety used in the assay. The detectable group can be any material having a detectable physical or chemical property. Thus, a label is any composition detectable by spectroscopic, photochemical, biochemical, electrical, optical or chemical means. A wide variety of labels may be used, with the choice of label depending on sensitivity required, ease of conjugation with the compound, stability requirements, available instrumentation, and disposal provisions. For a review of various labeling or signal producing systems that may be used, see U.S. Pat. No. 4,391,904.

Throughout the assays, incubation and/or washing steps may be required after each combination of reagents. Incubation steps can vary from about 5 seconds to several hours, optionally from about 5 minutes to about 24 hours. However, the incubation time will depend upon the assay format, antigen, volume of solution, concentrations, and the like. Usually, the assays will be carried out at ambient temperature, although they can be conducted over a range of temperatures, such as 10° C. to 40° C.

V. Kits

The invention also provides a kit comprising at least one unique polynucleotide tag with complementary strands as described above. The kit may also comprise a primer pair, i.e., a first primer that hybridizes to the first primer binding site on the first strand of each unique polynucleotide tag and a second primer that hybridizes to the second primer binding site on the second strand of each unique polynucleotide tag. In some embodiments, the first primer comprises a detectable label, such as, for example, a fluorescent label. In other embodiments, the second primer comprises a 5′ phosphate. In these embodiments, the kit can further include an exonuclease that digests DNA that has a 5′ phosphate.

Each polynucleotide tag is, in some embodiments, present in a vector (e.g., an adenoviral vector, a retroviral vector, or a lentiviral vector) that can integrate into a genome of a cell. In some embodiments, the kit further comprises a restriction enzyme that cleaves the polynucleotide tags at the unique restriction site. In some embodiments, the first polynucleotide tag is attached to a first protein binding moiety and the second polynucleotide tag is attached to a second protein binding moiety. For example, the first protein binding moiety and the second protein binding moiety can be antibodies. In some embodiments, the first polynucleotide tag comprises a first open circle probe and the second polynucleotide tag comprises a second open circle probe. The kit may also include a mixture of cell types, wherein each cell type comprises a unique oligonucleotide tag as described above. The cells may be any cells known in the art including, for example, yeast cells, bacterial cells, or mammalian cells. One of skill in the art will appreciate that the particular cell type is not a critical feature of the invention. Any cell type can be used provided that the particular cell type can be transfected by, i.e., is capable of being transfected by, a unique poly nucleotide tag as described above.

One or more additional containers may enclose elements, such as reagents or buffers, to be used in amplification assays. Additional components that may be present within such kits include instruction manuals.

### EXAMPLES

### Example 1

Materials and Methods

DNA constructs. The YEp24 vector, harboring the tetracycline resistance gene, was used as a template for the PCR-generated tags. The amplification of a 122 bp plasmid region (nucleotides −47 to +75 relative to the start codon of Tet^{R} ORF) was performed with one primer of constant length and the second one of variable length. The constant primer (cataaccaagcctatgcc) (SEQ ID NO:5) was complementary to +75 end and was common for all tags. The variable primers were used for the introduction of the unique and tag specific EcoRI site. They spanned the amplified region from the −47 end to a position 18 nucleotides downstream from the insertion site. (e.g. gGAATTCcggtagtttatcacagtt (SEQ ID NO:6)for tag 2 and gcgGAATTCgtagtttatcacagttaa (SEQ ID NO:7) for tag 4). The resulting PCR products were cloned into pUNI/V5-His-TOPO vector (Invitrogen, Carlsbad, Calif.) and all constructs were confirmed by sequencing. To subclone the tags into the yeast integration vector, the tag-containing pUNI/V5-His-TOPO vectors were linearized with NotI and cloned into the NotI site of pRS305. Integration of tags into the genomes of yeast strains was performed by transforming them with HpaI-linearized pRS305 plasmids harboring indicated tags. The plasmid integration was confirmed by the PCR amplification of tags from the yeast chromosomal DNAs and then EcoRI digestion.

Strains, media and inhibitors. All yeast strains used were Saccharomyces cerevisiae BY4743 derivatives purchased from Research Genetics (Invitrogen Corporation, Carlsbad, Calif.). Yeast strains were grown in the yeast extract/peptone/dextrose (YPD) medium supplemented with an inhibitor. Stock solutions of tunicamycin (Sigma, St. Louis, Mo.), latranculin B (Alexis, San Diego, Calif.), and staurosporine (Sigma, St. Louis, Mo.) were prepared by dissolving the compounds in DMSO at 0.2 mg/ml, 1 mg/ml, and 0.1 mM concentrations, respectively. Caffeine (Sigma, St. Louis, Mo.) was dissolved in water at 0.4 M concentration. Final concentrations of inhibitors in media were 8 mM, 2 μg/ml, 1 μM, and 0.4 μ/ml for caffeine, latrunculin B, staurosporine, and tunicamycin, respectively.

Measuring yeast growth. Individual yeast cultures were grown overnight in YPD medium to saturation. 1.5 μl of saturated culture was mixed with 100 μl of fresh YPD media containing the indicated inhibitors and filled into a well of Thermo Labsystems honeycomb microplate. The cultivation was performed in Microbiology Workstation Bioscreen C instrument (Thermo Labsystems, Helsinki, Finland) at 30° C. with continuous intensive shaking. O.D.600 was measured every hour for up to 2 days. To measure the changes in the composition of mixed cultures, equal volumes of 11 saturated strains were mixed, diluted 60-fold, and grown at 30° C. for 20 hours either in 1 ml media in falcon tubes or in 25 μl of media in a 384-well plate.

Tag amplification and capillary electrophoresis. Oligonucleotides Fluor-1 (6-Fam-gaagttatcggaggaattggctcgagg) and Pho-1 (Phosphate-aggagagggttagggataggcttaccg) were used for the amplification of tag DNAs. PCR reactions were performed in 50 μl reaction volume containing the 2 primers at a final concentration of 1 μM each and 2.5 units of Stoffel fragment AmpliTaq DNA polymerase (Applied Biosystems, Foster City, Calif.). After an initial 2 min denaturing step at 94° C., the reactions underwent 30 amplification cycles (94° C., 30 sec; 60° C., 30 sec; 72° C., 30 sec with a 6 sec increase in extension time per cycle) followed by a post-amplification extension step (72° C., 10 min). As a template for the PCR reactions, 100 ng of plasmid DNA, 700 ng of genomic DNA (extracted from yeast strains using MasterPure Yeast DNA purification kit; Epicentre, Madison, Wis.), or an equivalent to 0.5 μl of saturated culture of yeast protoplasts (prepared by using lyticase) (see, e.g., Chen et al., Biotechniques 19:744-746 (1995)) was used. The PCR products were incubated with λ-exonuclease for 1 h at 37° C. and then purified using MinElute PCR purification kit (QIAGEN Inc., Valencia, Calif.). After 1 h digestion with EcoRI, the PCR products were again purified with the same kit and adjusted to 70% formamide and 1 mM EDTA and analyzed by capillary electrophoresis on ABI Prism 3700 DNA analyzer (Applied Biosystems, Foster City, Calif.) using 3700 POP-5 polymer.

Data analysis. To access the raw fluorescence data collected by ABI Prism 3700 DNA analyzer, a Visual Basic program was used that extracted the electropherogram values and converted them into a text file format. Peak quantitation was then performed using Caesar Workstation 5.0 (SciBridge Software, Los Angeles, Calif.). In the majority of cases, the relative representation of each tag in the PCR product was calculated by dividing the area under the corresponding tag peak by the sum of all tag peak areas. For the measurement of different input amounts of tag 42, the average peak area of the other 10 tags was first calculated. The area value of tag 42 was then divided by this averaged value to give normalized tag 42 area.

### Example 2

Strain Tagging Strategy

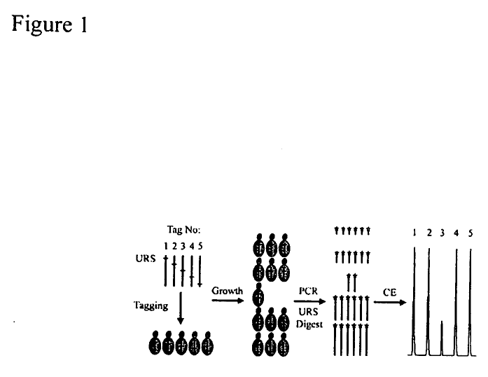

The Differential Tag Length Analysis (DTLA) method is designed to analyze quantitatively the composition of strain mixtures as outlined in FIG. 1. The tags are generated from a DNA fragment by introducing a unique EcoRI restriction site at variable but tag-specific positions. This results in almost identical tag DNA molecules differing only in the EcoRI site location. The tags are introduced into individual yeast strains, which are then mixed and grown under appropriate conditions. The replication of each tag is tightly controlled (via its integration into the strain genome) and thus the number of tag molecules per cell is constant throughout the whole course of cultivation. If the treatment results in a growth disadvantage for one or more strains in the mixture, the cells of those strains will be depleted. The relative loss of cells is accompanied by the same relative decrease in the number of corresponding tags. At the end of cultivation, all tags are amplified by multiplex PCR with a common pair of primers, one fluorescently labeled and the other phosphorylated at the 5′-end. The PCR products generated in this manner contained heteroduplex molecules formed by the annealing of complementary DNA strands of different tags that were resistant to EcoRI digestion. These heteroduplexes resulted in contaminating peaks appearing during capillary electrophoresis. Therefore, the PCR product was incubated with γ-exonuclease that selectively degrades the 5′-phosphorylated DNA strand ((see, e.g., Mitsis and Kwagh, Nucleic Acids Res. 27:3057-3063 (1999)). This treatment converted the PCR product into single-stranded fluorescent DNA molecules that could be efficiently digested by EcoRI restriction endonuclease to give fluorescent fragments of tag-specific lengths (see, e.g., Nishigaki et al., Nucleic Acids Res. 13:5747-5760 (1985) and Aggarwal, Curr. Opin. Struct. Biol. 5:11-19(1995)). After separation by capillary electrophoresis and peak quantitation, the proportional representation of tags should be equivalent to that of the tagged strains.

### Example 3

Tag Amplification Properties.

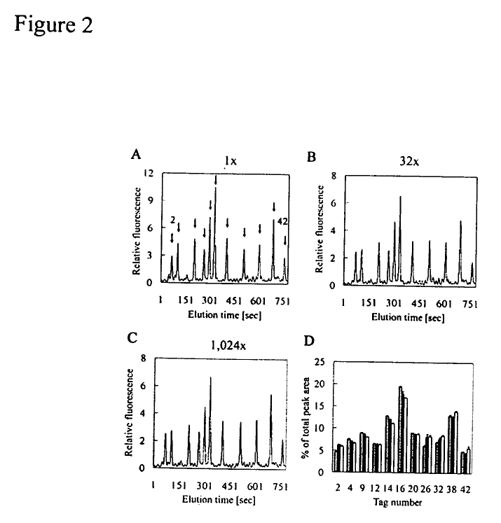

To verify that there are only small differences in tag amplification rates (the critical prerequisite for DTLA), PCR products from several reactions that underwent increasing numbers of template doublings were compared. During PCR amplification, fractions of slowly amplified tags would gradually decrease while those of rapidly amplified ones would increase. The extent of such a change would reflect the differences in amplification rates. A mixture of 11 tags with the EcoRI restriction site at nucleotide positions 2, 4, 9, 12, 14, 16, 20, 26, 32, 38, and 42 was used as the template for multiplex PCR. The PCR conditions were formulated such that the primers were the reaction extent-limiting component. The concentration of DNA polymerase and dNTPs, and the number of thermal cycles were in excess to allow the reaction to continue until primer exhaustion. Such a set up resulted in a constant amount of PCR product independent of starting amount of template. Thus, different starting template concentrations would determine the number of template doublings. The initial tag mixture was then diluted 32× and 1,024× to create 3 separate template stocks. Each consecutive template dilution resulted in 5 additional DNA doubling cycles with the maximum difference of 10 cycles between the most concentrated and most diluted stock. The tag PCR profiles for the different template dilutions are shown in FIG. 2A-C. As expected, the tags' fractional representation in the final product did not change significantly with the template dilution (FIG. 2D). Tags 2, 12, 20, and 38 were amplified with an average rate. Tags 26, 32, and 42 were slightly expanding while tags 4, 9, 14, and 16 were depleted to some extent. Overall, very limited change in each tag's representation was observed (less than 15% of its original value even after 1,024-fold amplification), implying that all tags were amplified at an almost identical rate.

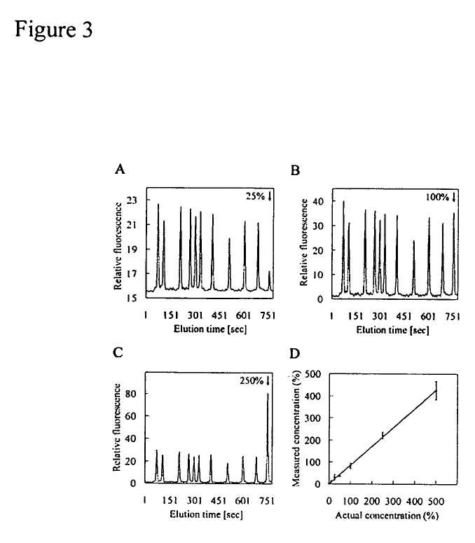

To characterize the dynamic range of DTLA, an approximately equimolar mixture of 10 tags (tags 2, 4, 9, 12, 14 16, 20, 26, 32, and 38) was made. It was supplemented with different quantities of the 11^{th} tag (tag 42), at 0%, 10%, 25%, 50%, 100%, 250%, and 500% relative to the concentration of other tags. The mixtures were analyzed by DTLA and typical electropherograms of the assay are shown in FIG. 3A-C. The measured concentration of tag 42 in each condition was first normalized and then plotted against the relative input concentrations of tag 42. The resulting curve showed excellent linearity across the tested range (FIG. 3D). Comparable results were also obtained when characterizing the other tags. All of the above observations suggest that DLTM is a quantitative and robust method for the measurement of mixture composition.

### Example 4

Selection of Yeast Strains Comprising Model Array.

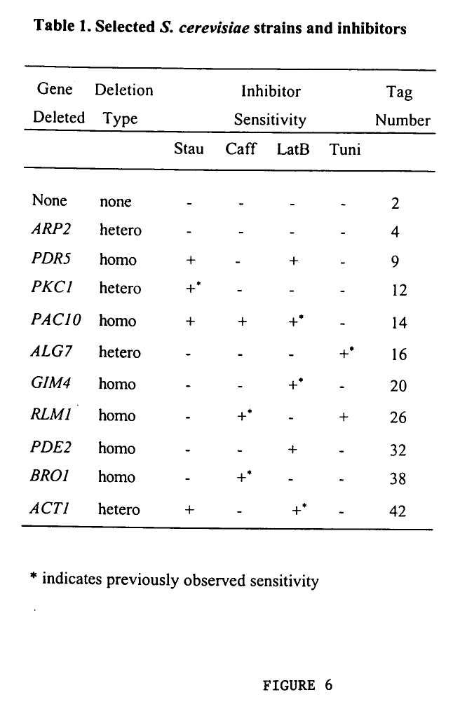

To follow the behavior of a multi-strain culture grown under different external conditions, 10 mutant yeast strains from the comprehensive deletion set with previously characterized drug sensitivities were chosen. The selection was based on our interest in the cellular pathways affected in the mutants and the availability of small molecule inhibitors for those pathways (see, e.g., Ayscough et al., J. Cell Biol. 137:399-416 (1997); Giaver et al., Nat. Genet. 21:278-283 (1999); Hampsey, Yeast 13:1099-1133 (1997); Watanabe et al. Mol. Cell. Biol. 17:2615-2623 (1997); and Geissler et al., EMBO J. 17:952-966 (1998)). Strains included mutants with the deletion of genes participating in cytoskeletal organization (ACT1, ARP2, GIM4, and PAC10), cell wall integrity signaling (PKC1 and RLM1), nutrient sensing (BRO1 and PDE2), N-glycosylation (ALG7), and drug efflux (PDR5). In the case of essential genes (ACT1, ALG7, ARP2, and PKC1), heterozygous diploid strains lacking only one of the two gene copies were used; the remaining strains were homozygous diploid deletions. The 10 mutants were supplemented with the wild-type strain to give the final array. This array was then tested with caffeine, latrunculin B, staurosporine, and tunicamycin (FIG. 6, known drug sensitivities indicated as a plus with an asterisk).

### Example 5

Competitive Interactions among the Strains in a Mixed Culture

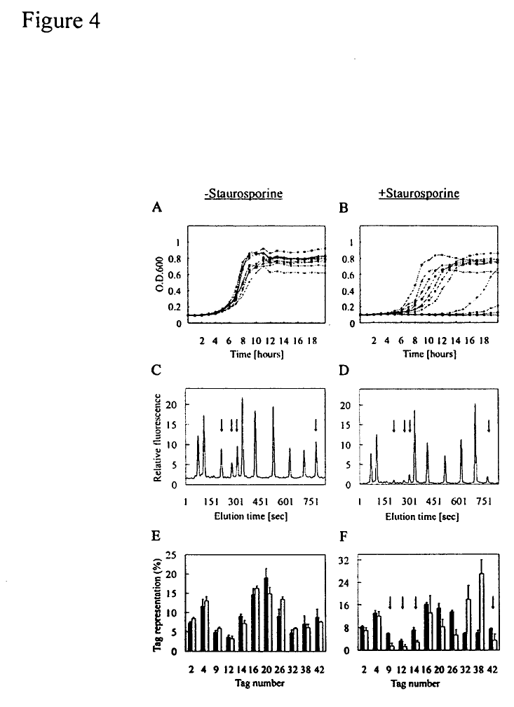

The competition among mixed yeast strains can induce changes in growth behavior not observed during separate cultivation. To assess the significance of such effects, the strains' growth in mixture was first characterized. The 11 selected yeast strains were modified by integrating the 11 above described tags into their genomes (FIG. 6) and the strain mixture was grown in medium without an inhibitor. The tag compositions of culture at the beginning and end of cultivation were compared (FIG. 4E). The most dramatic increase in representation was observed for rlm1Δ/rlm1Δ mutant (tag 26, increased by 50%). The wild type, arp2Δ/ARP2, pdr5Δ/pdr5Δ, alg7Δ/ALG7 strains were also enriched by 9% and pde2Δ/pde2Δ strain increased by 20%. The expansion of this group in the mixture came at the expense of the rest of the deletion mutants with pac10Δ/pac10Δ and gim4Δ/gim4Δ mutants (decreased by 25% and 28% respectively) as the most depleted strains. Interestingly, both PAC10 and GIM4 encode subunits of the Gim4 complex participating in the formation of functional alpha- and gamma-tubulin. The same growth defect of both deletions suggests that tag profiles reflect the true strain compositions.

### Example 6

Staurosporine Sensitivities of Selected Strains

The presence of staurosporine in the medium dramatically changed the behavior of individually grown strains (FIG. 4A vs. 4B). Based on the growth properties, the strains could be clearly classified as either sensitive (ACT1/act1Δ, pac10Δ/pac10Δ, pdr5Δ/pdr5Δ, and PKC1/pkc1Δ strains) or insensitive (wild-type, alg7Δ/ALG7, arp2Δ/ARP2, bro1Δ/bro1Δ/bro1Δ, gim4Δ/gim4Δ, pde2Δ/pde2Δ, and rlm1Δ/rlm1Δ strains). Strains from the insensitive set also exhibited larger variations in their growth than in the absence of staurosporine. Upon examination within the insensitive group, gim4Δ/gim4Δ and rlm1Δ/rlm1Δ mutants (FIG. 4A, B; red squares and red circles, respectively) displayed the largest degree of inhibition. The bro1Δ/bro1Δ and pde2Δ/pde2Δ mutants (green squares and red triangles, respectively) were not significantly affected by this inhibitor and became the fastest growers (FIG. 4B).

Addition of staurosporine to the mixed culture caused nearly complete elimination of the 4 tag peaks representing the staurosporine sensitive strains (arrows in FIG. 4C, D). Detailed quantitative analysis showed that these 4 strains (pdr5Δ/pdr5Δ, pkc1Δ/PKC1, pac10Δ/pac10Δ, and act1Δ/ACT1) were depleted by 75%, 61%, 60%, and 53%, respectively, relative to the non-treated culture (FIG. 4F). There was also significant depletion of the two slowest growing strains from the staurosporine insensitive group, rlm1Δ/rlm1Δ (decrease by 60%) and gim4Δ/gim4Δ (decrease by 44%). The most resistant strains to staurosporine were bro1Δ/bro1Δ (increase by 340%) and pde2Δ/pde2Δ (increase by 200%) mutants. Overall, the inhibitory pattern induced by staurosporine in mixed culture was identical to that observed for the strains growing individually.

### Example 7

Other Drug Sensitivities.

Similarly, the growth of the 11 strains in the presence of the other 3 inhibitors by DTLA was examined. During individual growth, the caffeine sensitive strains were bro1Δ/bro1Δ, pac10Δ/pac10Δ, and rlm1Δ/rlm1Δ mutants. Latrunculin B inhibited the growth of act1Δ/ACT1, gim4Δ/gim4Δ, pac10Δ/pac10Δ, pde2Δ/pde2Δ, and pdr5Δ/pdr5Δ deletion strains. Tunicamycin slowed the growth of alg7Δ/ALG7 and rlm1Δ/rlm1Δ mutants (FIG. 6). In summary, all previously reported mutant sensitivities were confirmed and some novel inhibitor-induced behaviors were observed.

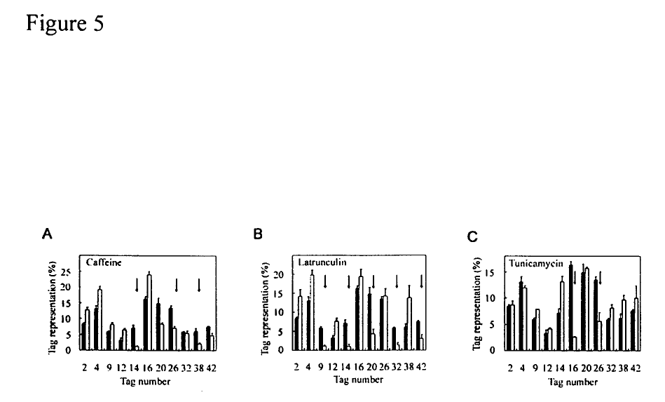

In mixed culture, caffeine induced decreased growth of all 3 caffeine-sensitive strains mentioned above and also the 2 slowest growers from the insensitive group (pde2Δ/pde2Δ and act1Δ/ACT1). The expansion of the insensitive strains was distributed more evenly than for staurosporine, with pkc1Δ/PKC1Δ mutant making the largest gain (increase by 97%; FIG. 5A). Treatment of the mixed culture with latrunculin B resulted in the inhibition of the same subset of mutants as was observed for individual growth (pdr5Δ/pdr5Δ, pac10Δ/pac10Δ, gim4Δ/gim4Δ, pde2Δ/pde2Δ, and act1Δ/ACT1Δ mutants; FIG. 5B). Each of the 5 sensitive strains was reduced to less than 50% when compared with the non-treated culture. The expansion of insensitive strains was nearly uniform, with bro1Δ/bro1Δ mutant benefiting the most (increase by 123%). The last inhibitor, tunicamycin, also showed complete overlap between sensitive strains from individually grown and mixed cultures (alg7Δ/ALG7 and rlm1Δ/rml1Δ, 83% and 59% reduction, respectively; FIG. 5C). The best growing strain from this experiment was the pac10Δ/pac10Δ mutant that increased its representation in the mixture by 82%. Very similar results for all tested inhibitors were also obtained when the array was cultivated in volumes as low as 25 μl.

A remarkable agreement was observed a between the data obtained through individual versus mixed cultivations for the inhibitors tested. This demonstrates that DTLA is highly suitable for the precise measurement of dynamically evolving complex systems.

Phenotypic profiling of mutant yeast and bacterial strains in the presence of small molecule inhibitors has proven to be a powerful tool for both novel gene discovery and characterization of interesting compounds (see, e.g., Hung et al., Chem. Biol. 8:623-639 (1996); Giaver et al., Nat. Genet. 21:278-283 (1999); De Backer et al., Nat. Biotechnol. 19:235-241 (2001); and DeVito et al., Nat. Biotechnol. 20:478-483 (2002)). To accelerate the discovery process for arrays of such mutants, DTLA was developed to measure the growth of all strains in a mixture while cultivated in volumes as low as 25 μl. DTLA utilizes DNA tags of novel design that allow a constant amplification rate of all tags and the representation of tags in the product reflects the strain composition of the mixture. Our expectation of low signal distortions was fully confirmed by the direct measurement of amplification rates during multiplex PCR of the 11 tag mixture with the change in representation of any tag being less than 15% after 1,024-fold amplification. The method was highly reproducible and also exhibited very good linear dose-response within at least a 50-fold concentration range.

To test DTLA with an array of yeast strains, 10 Saccharomyces cerevisiae mutants with previously characterized drug sensitivities were chose. The set was supplemented with the wild-type yeast strain. Evaluation of growth in a mixture without an inhibitor was used as the initial test of DTLA and also served as the baseline for the subsequent measurement of inhibitory effects. Similar to the separately cultured strains, the growth differences in the mixture were minor with the exception of the rlm1Δ/rlm1Δ mutant, which exhibited unexpectedly vigorous growth in the mixture. Rlm1p is a MADS-box transcription factor that functions in cell wall integrity signaling. However, it is unclear why the strain shows such vigorous growth under competitive conditions.

Comparison of inhibitor sensitive growth between strains cultured individually and in a mixture showed a remarkable degree of agreement. All strains sensitive to a particular inhibitor when grown individually were also dramatically depleted from the mixture when that inhibitor was present. In addition, some of the mutants classified as insensitive when grown individually (gim4Δ/gim4Δ and rlm1Δ/rlm1Δ mutants for staurosporine and gim4Δ/gim 4Δ and act1Δ/ACT1 mutants for caffeine) exhibited sensitivity in the mixture (FIG. 4B). This observation can be explained by the fact that the growth competition in a mixture expands subtle growth differences observed during individual growth. Indeed, all of 4 the above cases were the slowest growing strains from a corresponding insensitive group. In a similar manner, bro1Δ/bro1Δ and pde2Δ/pde2Δ mutants dramatically increased their representation after growth in the mixture when staurosporine was present. This further demonstrates that DTLA is a reliable, accurate, and a very sensitive method.

One goal of this study was to develop a method for multiplexing yeast growth measurement in minimal cultivation volumes that can be performed in a fully automated mode. While its feasibility with the set of 11 strains has been demonstrated, the upper limit for the number of strains that can be grown in a mixture is determined by the resolving power of the capillary electrophoresis instrument. For example, an ABI3700 DNA analyzer in combination with POP-5 separation polymer would be able to separate up to 700 nucleotides, corresponding to ˜700 strains. New applications of DTLA will undoubtedly be found for multiplexing other assays that can be linked to DNA tags, e.g. the measurement of cell proliferation in mammalian tissue culture for identification of anti-proliferation compounds, or multiplexing of quantitative immuno-PCR for simultaneous quantitation of multiple antigens in a mixture. Therefore, DTLA should accelerate the identification of phenotypes associated with mutants lacking studied genes, and facilitate the discovery of biologically active small molecules both as research tools and therapeutic agents.

It is understood that the examples and embodiments described herein are for illustrative purposes only and that various modifications or changes in light thereof will be suggested to persons skilled in the art and are to included within the spirit and purview of this application and are considered within the scope of the appended claims. All publications, patents, and patent applications cited herein are hereby incorporated by reference in their entirety for all purposes.

1. A method for determining the relative prevalence of one or more cell types in a mixture of cell types in a culture, the method comprising:
a) providing at least one cell of a first cell type and at least one cell of at least a second cell type, wherein the first cell type comprises a first polynucleotide tag and the second cell type comprises a second polynucleotide tag, and further wherein each polynucleotide tag comprises:
i) a first primer binding site on a first strand and a second primer binding site on a second strand, wherein the second strand is complementary to the first strand, and wherein:
1) a primer that hybridizes to the first primer binding site on the first polynucleotide tag also hybridizes to the first primer binding site on the second polynucleotide tag; and
2) a primer that hybridizes to the second primer binding site on the first polynucleotide tag also hybridizes to the second primer binding site on the second polynucleotide tag;
ii) a unique restriction site at a preselected position relative to the first primer binding site, wherein the position of the unique restriction site in the first polynucleotide tag differs from the position of the unique restriction site in the second polynucleotide tag;
b) incubating the cells in a medium and under conditions suitable for proliferation of the cells; and
c) detecting relative prevalence of one or more of the cell types by:
(i) amplifying the polynucleotide tags with a pair of primers that hybridize to the first and second primer binding sites, thereby producing an amplified product corresponding to a polynucleotide tag if the polynucleotide tag is present;
(ii) cleaving the amplified products with a restriction enzyme that cleaves the amplified products at the unique restriction site, thereby forming cleaved amplification products; and
(iii) detecting the cleaved amplification products, whereby the amount of a first cleaved amplification product corresponding to the first polynucleotide tag is correlated with the relative prevalence of the first cell type and the amount of a second cleaved amplification product corresponding to the second polynucleotide tag is correlated with the relative prevalence of the second cell type.

2. The method of claim 1, wherein the cells are yeast cells.

3. The method of claim 1, wherein the cells express two interacting proteins and proliferation of the cells depends upon the interaction between the proteins.

4. The method of claim 1, wherein the cells are mammalian cells.

5. The method of claim 4, wherein the mammalian cells are cancer cells.

6. The method of claim 1, wherein the polynucleotide tags are integrated into the genomes of the first and second cell types.

7. The method of claim 1, wherein the method comprises performing the amplification on a sample of purified genomic DNA obtained from the cells.

8. The method of claim 1, wherein the method comprises performing the amplification on a cell lysate.

9. The method of claim 1, wherein the first inserted polynucleotide sequence comprises the sequence set forth in SEQ ID NO:1 and the second inserted polynucleotide sequence comprises the sequence set forth in SEQ ID NO:2.

10. The method of claim 1, wherein the restriction enzyme recognition site is specific for EcoRI.

11. The method of claim 1, wherein at least one primer in the pair of primers is labeled.

12. The method of claim 1, wherein both primers are labeled.

13. The method of claim 11, wherein the second primer in the pair of primers comprises a 5′ phosphate.

14. The method of claim 13, wherein the method further comprises contacting the amplified reaction products with an exonuclease that degrades a polynucleotide strand that comprises the primer that comprises the 5′ phosphate prior to contacting the amplified reaction products with the restriction enzyme.

15. The method of claim 1, wherein the relative prevalence of at least three cell types is detected, and wherein each cell type comprises a polynucleotide tag in which the position of the unique restriction site in the polynucleotide tag present in one cell type differs from the position of the unique restriction site in the polynucleotide tag present in other cell types.

16. The method of claim 1, wherein the relative prevalence of at least ten cell types is detected, and wherein each cell type comprises a polynucleotide tag in which the position of the unique restriction site in the polynucleotide tag present in one cell type differs from the position of the unique restriction site in the polynucleotide tag present in other cell types.

17. The method of claim 1, wherein the cells are contacted with a potential modulator of cell proliferation.

18. The method of claim 17, wherein the potential modulator of cell proliferation is a small organic molecule.

19. The method of claim 1, wherein the cells are contacted with a potential modulator of cell death.

20. The method of claim 19, wherein the potential modulator of cell death is an apoptosis inducer.

21. The method of claim 1, wherein at least one gene in at least a first cell type is expressed at a level that differs from expression of the gene in other cell types present.

22. The method of claim 21, wherein cells of the first cell type comprise a mutation that alters expression of the gene.

23. The method of claim 21, wherein cells of the first cell type comprise an expression construct from which is transcribed mRNA transcripts that correspond to those transcribed from the gene present in the genome of the cell, thereby causing the cells of the first cell type to comprise a higher level of mRNA transcripts that correspond to the gene than other cell types.

24. The method of claim 21, wherein cells of the first cell type comprise a double-stranded RNA molecule that comprises a first polynucleotide sequence that is identical to a target region on the gene, and a second polynucleotide sequence that is complementary to the first polynucleotide sequence, wherein the double-stranded RNA molecule inhibits expression of the gene in cells of the first cell type.

25. The method of claim 1, wherein the cells are in a well of a microtiter plate.

26. A method of detecting proliferation of a mixture of cell populations in a single vessel, the method comprising:
providing at least two cell populations, wherein each cell population comprises a unique inserted polynucleotide sequence at a preselected unique position; and
detecting proliferation of the cell populations by:
(a) amplifying the inserted polynucleotide sequences with a primer pair specific for the inserted polynucleotide sequences, wherein the primer pair amplifies any of the inserted polynucleotide sequences that are present, thereby producing amplified products;
(b) cleaving the amplified products with a restriction enzyme, thereby forming cleaved amplification products; and
(c) detecting the cleaved amplification products, whereby detection of the cleaved amplification products detect the proliferation of each cell population.

27. A method for screening a library of compounds to identify those compounds that alter the relative prevalence of one or more cell types in a mixture of cell types, the method comprising:
a) providing at least one cell of a first cell type and at least one cell of at least a second cell type, wherein cells of the first cell type comprise a first polynucleotide tag and cells of the second cell type comprise a second polynucleotide tag, and further wherein each polynucleotide tags comprises:
i) a first primer binding site on a first strand and a second primer binding site on a second strand, wherein the second strand is complementary to the first strand, and wherein:
1) a primer that hybridizes to the first primer binding site on the first polynucleotide tag also hybridizes to the first primer binding site on the second polynucleotide tag; and
2) a primer that hybridizes to the second primer binding site on the first polynucleotide tag also hybridizes to the second primer binding site on the second polynucleotide tag;
ii) a unique restriction site at a preselected position relative to the first primer binding site, wherein the position of the unique restriction site in the first polynucleotide tag differs from the position of the unique restriction site in the second polynucleotide tag;
b) contacting cells with a member of a library of compounds and incubating the cells under conditions suitable for proliferation of the cells; and
c) determining relative prevalence of one or more of the cell types by:
(i) amplifying the polynucleotide tags with a pair of primers that hybridize to the first and second primer binding sites, thereby producing an amplified product corresponding to a polynucleotide tag if the polynucleotide tag is present;
(ii) cleaving the amplified products with a restriction enzyme that cleaves the amplified products at the unique restriction site, thereby forming cleaved amplification products; and
(iii) detecting the cleaved amplification products, whereby the amount of a first cleaved amplification product corresponding to the first polynucleotide tag is correlated with the relative prevalence of the first cell type in the presence of the compound of step (b), and the amount of a second cleaved amplification product corresponding to the second polynucleotide tag is correlated with the relative prevalence of the second cell type in the presence of the compound of step (b).

28. The method of claim 27, wherein the cells are in a well of a microtiter plate.

29. The method of claim 27, wherein the microtiter plate is a 96, 384 or 1536 well microtiter plate.

30. The method of claim 27, wherein the cells are in a vessel and at least one test gene in at least a first cell type is expressed at a level that differs from expression of the test gene in other cell types present in the vessel, and
whereby a change in the amount of the cleaved amplification product corresponding to the first cell type in a first vessel compared to the amount of the cleaved amplification product corresponding to the first cell type in at least a second vessel indicates that the test gene in the first cell type encodes a gene product that is modulated by a compound present in the first vessel.

31. The method of claim 30, wherein cells of the first cell type comprise a mutation that alters expression of the test gene.

32. The method of claim 30, wherein cells of the first cell type comprise an expression construct from which is transcribed mRNA transcripts that correspond to those transcribed from the test gene present in the genome of the cell, thereby causing the cells of the first cell type to comprise a higher level of mRNA transcripts that correspond to the test gene than other cell types.

33. The method of claim 30, wherein cells of the first cell type comprise a double-stranded RNA molecule that comprises a first polynucleotide sequence that is identical to a target region on the test gene, and a second polynucleotide sequence that is complementary to the first polynucleotide sequence, wherein the double-stranded RNA molecule inhibits expression of the test gene in cells of the first cell type.

34. The method of claim 27, wherein the compound alters the relative prevalence of a cell type by modulating proliferation of the cell type.

35. The method of claim 27, wherein the compound alters the relative prevalence of a cell type by modulating death of the cell type.

36. A mixture of cells comprising two or more cell types, wherein each cell type comprises a polynucleotide tag comprising:
i) a first primer binding site on a first strand and a second primer binding site on a second strand, wherein the second strand is complementary to the first strand, and wherein:
1) a primer that hybridizes to the first primer binding site on the first polynucleotide tag also hybridizes to the first primer binding site on the second polynucleotide tag; and
2) a primer that hybridizes to the second primer binding site on the first polynucleotide tag also hybridizes to the second primer binding site on the second polynucleotide tag;
ii) a unique restriction site at a preselected position relative to the first primer binding site, wherein the position of the unique restriction site in the first polynucleotide tag differs from the position of the unique restriction site in the second polynucleotide tag.

37. The mixture of claim 36, comprising five or more cell types.

38. The mixture of claim 36, comprising 10 or more cell types.

39. The mixture of claim 36, comprising 100 or more cell types.

40. The mixture of claim 36, wherein the cells are yeast cells.

41. The mixture of claim 36, wherein the cells express a pair of interacting proteins and cell proliferation is dependent upon the interaction between the proteins.

42. The mixture of claim 36, wherein the cells are mammalian cells.

43. The mixture of claim 42, wherein the mammalian cells are cancer cells.

44. A method for detecting the presence or absence of antigens in a sample, the method comprising:
a) contacting a sample with at least a first and a second detection reagent, wherein the first detection reagent comprises: a) a binding moiety specific for a first antigen, and b) a first polynucleotide tag; and the second detection reagent comprises: a) a binding moiety specific for a second antigen, and b) a second polynucleotide tag,
wherein each polynucleotide tag comprises:
i) a first primer binding site on a first strand and a second primer binding site on a second strand, wherein the second strand is complementary to the first strand, and wherein:
1) a primer that hybridizes to the first primer binding site on the first polynucleotide tag also hybridizes to the first primer binding site on the second polynucleotide tag; and
2) a primer that hybridizes to the second primer binding site on the first polynucleotide tag also hybridizes to the second primer binding site on the second polynucleotide tag;
ii) a unique restriction site at a preselected position relative to the first primer binding site, wherein the position of the unique restriction site in the first polynucleotide tag differs from the position of the unique restriction site in the second polynucleotide tag;
b) separating the detection reagents that bind to an antigen in the sample from detection reagents that do not bind to the antigen; and
c) detecting the presence or absence of the bound first and second detection reagents by:
(i) amplifying the polynucleotide tags with a pair of primers that hybridize to the first and second primer binding sites, thereby producing an amplified product corresponding to a polynucleotide tag if the polynucleotide tag is present;
(ii) cleaving the amplified products with a restriction enzyme that cleaves the amplified products at the unique restriction site, thereby forming cleaved amplification products; and
(iii) detecting the cleaved amplification products, whereby the amount of a first cleaved amplification product corresponding to the first polynucleotide tag is correlated with the amount of the first antigen and the amount of a second cleaved amplification product corresponding to the second polynucleotide tag is correlated with the amount of the second antigen in the sample.

45. The method of claim 44, wherein one or more of the binding moieties comprise antibodies.

46. The method of claim 44, wherein the polynucleotide tags each comprise an open circle probe that is attached to the binding moiety by hybridization between the open circle probe and a target probe that is attached to the corresponding binding moiety.

47. A kit that comprises:
a) at least first polynucleotide tag and a second polynucleotide tag, and further wherein each polynucleotide tag comprises:
i) a first primer binding site on a first strand and a second primer binding site on a second strand, wherein the second strand is complementary to the first strand, and wherein:
1) a primer that hybridizes to the first primer binding site on the first polynucleotide tag also hybridizes to the first primer binding site on the second polynucleotide tag; and
2) a primer that hybridizes to the second primer binding site on the first polynucleotide tag also hybridizes to the second primer binding site on the second polynucleotide tag;
ii) a unique restriction site at a preselected position relative to the first primer binding site, wherein the position of the unique restriction site in the first polynucleotide tag differs from the position of the unique restriction site in the second polynucleotide tag;
b) a first primer that hybridizes to each of the polynucleotides at the first primer binding site and a second primer that hybridizes to a complementary strand of each of the polynucleotides at the second primer binding site.

48. The kit according to claim 47, wherein the first primer comprises a detectable label.

49. The kit according to claim 48, wherein the label is a fluorescent label.

50. The kit according to claim 48, wherein the second primer comprises a 5′ phosphate.

51. The kit according to claim 50, wherein the kit further comprises an exonuclease.

52. The kit according to claim 47, wherein each polynucleotide tag is present in a vector that can integrate into a genome of a cell.

53. The kit according to claim 52, wherein the vector is an adenoviral vector, a retroviral vector, or a lentiviral vector.

54. The kit according to claim 47, wherein the kit further comprises a restriction enzyme that cleaves the polynucleotide tags at the unique restriction site.

55. The kit according to claim 47, wherein the kit comprises at least three polynucleotide tags in which the position of the unique restriction site in each polynucleotide tag differs from the position of the unique restriction site in the other polynucleotide tags in the kit.

56. The kit according to claim 55, wherein the kit comprises at least ten polynucleotide tags in which the position of the unique restriction site in each polynucleotide tag differs from the position of the unique restriction site in the other polynucleotide tags in the kit.

57. The kit according to claim 47, wherein the first polynucleotide tag is attached to a first protein binding moiety and the second polynucleotide tag is attached to a second protein binding moiety.

58. The kit according to claim 57, wherein the first protein binding moiety and the second protein binding moiety are antibodies.

59. The kit according to claim 47, wherein the first polynucleotide tag comprises a first open circle probe and the second polynucleotide tag comprises a second open circle probe.


SEQUENCE LISTING (9 sequences)

SEQ ID NO: 1 - DNA, 210 bp, Artificial Sequence
  Description of Artificial Sequencefirst inserted polynucleotide tag sequence
gaagttatcggaggaattggctcgaggaattgcccttgaattcgcggtagtttatcacagttaaattgctaacgcagtcaggcaccgtgtatgaaatctaacaatgcgctcatcgtcatcctcggcaccgtcaccctggatgctgtaggcataggcttggttatgaagggcaattcgggagctcggtaagcctatccctaaccctctcct

SEQ ID NO: 2 - DNA, 210 bp, Artificial Sequence
  Description of Artificial Sequencesecond inserted polynucleotide tag sequence
gaagttatcggaggaattggctcgaggaattgcccttgcgggaattctagtttatcacagttaaattgctaacgcagtcaggcaccgtgtatgaaatctaacaatgcgctcatcgtcatcctcggcaccgtcaccctggatgctgtaggcataggcttggttatgaagggcaattcgggagctcggtaagcctatccctaaccctctcct

SEQ ID NO: 3 - DNA, 27 bp, Artificial Sequence
  Description of Artificial Sequence oligonucleotide tag PCR amplification 5′ primer
gaagttatcggaggaattggctcgagg

SEQ ID NO: 4 - DNA, 27 bp, Artificial Sequence
  Description of Artificial Sequence oligonucleotide tag PCR amplification 3′ primer
aggagagggttagggataggcttaccg

SEQ ID NO: 5 - DNA, 18 bp, Artificial Sequence
  Description of Artificial SequencePCR amplification constant primer
cataaccaagcctatgcc

SEQ ID NO: 6 - DNA, 25 bp, Artificial Sequence
  Description of Artificial SequencePCR amplification variable primer for tag 2
ggaattccggtagtttatcacagtt

SEQ ID NO: 7 - DNA, 27 bp, Artificial Sequence
  Description of Artificial SequencePCR amplification variable primer for tag 4
gcggaattcgtagtttatcacagttaa

SEQ ID NO: 8 - DNA, 27 bp, Artificial Sequence
  Description of Artificial SequencePCR amplification oligonucleotide Fluor-1
naagttatcggaggaattggctcgagg

SEQ ID NO: 9 - DNA, 27 bp, Artificial Sequence
  Description of Artificial SequencePCR amplification oligonucleotide Pho-1
nggagagggttagggataggcttaccg